(19) United States
(12) Patent Application Publication
(10) Pub. No.: US 2025/0316344 A1
(43) Pub. Date: Oct. 9, 2025

(54) SYSTEMS AND METHOD FOR QUERY-BASED RANDOM ACCESS INTO VIRTUAL CHEMICAL COMBINATORIAL SYNTHESIS LIBRARIES

(71) Applicant: Atomwise Inc., San Francisco, CA (US)

(72) Inventors: Aryan Pedawi, San Francisco, CA (US); Henry van den Bedem, San Francisco, CA (US); Chaoyi Chang, San Francisco, CA (US); Brandon Anderson, San Francisco, CA (US); Pawel Gniewek, San Francisco, CA (US)

(21) Appl. No.: 18/864,857

(22) PCT Filed: May 16, 2023
(86) PCT No.: PCT/US23/67079

§ 371 Date: Nov. 11, 2024

Related U.S. Application Data
(60) Provisional application No. 63/342,574, filed on May 16, 2022.

(51) Int. Cl. G16C 20/64; G16C 20/40

(52) U.S. Cl. CPC G16C 20/64; G16C 20/40

(57) ABSTRACT

Systems and methods for querying a combinatorial synthesis library comprising a plurality of compounds and representing a plurality of reaction types, where each reaction type maps to a plurality of reactants, and each reactant maps to a plurality of synthons, accepts a query in the form of a single graph into a molecular encoder model, thereby obtaining a query vector. The query vector is inputted into a reaction query generator model thereby obtaining a first reaction type and a first plurality of reactants. A synthon is determined for each reactant by inputting the reactant into a synthon query generator model. A set of synthons is therefore determined, each corresponding to a reactant in the first plurality of reactants. A molecular structure in the combinatorial synthesis library is identified that includes the set of synthons arranged in accordance with a synthesis rule associated with the first reaction type.

### CROSS REFERENCE TO RELATED PATENT APPLICATION

This application claims priority to U.S. Provisional patent application No. 63/342,574, entitled “GRAPH GENERATIVE MODEL FOR COMBINATORIAL SYNTHESIS LIBRARIES,” filed May 16, 2022, which is hereby incorporated by reference.

### TECHNICAL FIELD

The disclosure relates generally to screening combinatorial synthesis libraries for target analogues.

### BACKGROUND

Virtual high throughput screening (vHTS) [47] has gained significant traction in early-stage drug discovery, owing in no small part to make-on-demand chemical libraries utilizing a combinatorial synthesis construction. These combinatorial synthesis libraries (CSLs) enable access to ultra-large swaths of chemical space from a considerably smaller set of chemically accessible building blocks that can be combined according to known synthesis routines. In recent years, these libraries have grown from millions, to billions, and now to trillions of compounds [22, 33, 44, 58]. As a result, virtual chemical libraries are quickly approaching a size beyond that which permits explicit enumeration, presenting new challenges for virtual screening. For example, the Enamine REadily Accessible (REAL) libraries [20] leverage off-the-shelf molecular building blocks and parallel synthesis, permitting lead times on the order of a few weeks and ushering in an era of ever-decreasing latency between in silico and in vitro high throughput screening.

As a result of the combinatorial explosion these constructions enable, early-stage drug discovery has now “crossed the Rubicon” into the non-enumerative regime. This presents new challenges for in silico hit discovery and optimization, which rely on screening explicitly enumerated compounds. These methods are ill suited to the non-enumerative setting, scaling linearly with the number of compounds.

Virtual high throughput screening and enumeration. Often, the first step in a vHTS campaign is preparing a compound library for subsequent use [1, 17]. While compound sampling and scoring techniques have been developed [18], these approaches nevertheless rely on an exhaustively accessible library. An exception is the virtual synthon hierarchical enumeration screening (V-SYNTHES) approach [44], which leverages the modular nature of parallel synthesis libraries. However, by design, V-SYNTHES does not permit query-based random access. On the other hand, SpaceMACS [45] and SpaceLight [4] can provide query-based access to modular libraries by decomposing the query into fragments and matching those by similarity search to synthons in the library. In parallel to these efforts, machine learning has received significant attention in vHTS: for predicting activity scores given docked conformations [15, 43, 57], predicting activity scores given a ligand and protein separately (undocked) [38, 56], and in improving or altogether replacing classical molecular docking with machine learning approaches [39, 51, 52].

Deep learning approaches to molecular generation. De novo drug design has assumed an increasingly prominent role in identifying novel chemical matter in drug discovery campaigns [10, 36, 50, 54]. The two dominant neural network-based paradigms for molecular generation are text-based and graph-based generative models. Early work in text-based generative models (also called chemical language models) applied recurrent neural networks to SMILES strings [16, 46]. Although these methods have shown a great deal of promise and spurred interest in molecular generation within the ML community, they are not guaranteed to produce valid SMILES strings. Approaches utilizing grammar constraints of SMILES notation have been proposed to improve validity [12, 29]; separately the recently proposed SELFIES notation [28, 37] guarantees validity and has seen increased adoption as such. In both cases, however, there remain known drawbacks to modeling with such text-based representations of chemical matter (e.g., subjectivity, similar molecular structures having possibly large edit distances).

For some applications, it is of interest to utilize generative models that can fit molecular databases, permitting the navigation of these databases via the fitted model. The ability of language models to fit molecular databases was investigated in a prior study [2] that applied deep language models to GDB-13 [7], a database of 975,000,000 compounds formed by fully enumerating molecules up to 13 atoms of element types C, N, O, S, and Cl, subject to simple chemical stability and synthetic feasibility rules. The authors trained on 0.1% of the total library and find that the model was capable of covering roughly 70% of compounds in the GDB-13 library. Furthermore, the language model they trained generates compounds not satisfying the GDB-13 construction in approximately 15% of cases.

Graph generative models have received significant attention in recent years as an alternative to their text-based counterparts. The earliest of these models focused on generating graphs of a constant size in a single shot [48] or generating graphs of arbitrary size autoregressively, one atom or bond at a time [32, 42, 60, 35]. These approaches also struggle to reliably produce chemically valid molecules and encounter difficulties with large molecular graphs.

In an effort to address both points, fragment-based graph generative models have been proposed and are growing in popularity [23, 24, 25, 27]. These models have the advantage of guaranteeing chemical validity by decomposing molecules into valid sub-components and explicitly disallowing actions which yield invalid combinations of fragments. Such explicit validity checks can be performed on every action, at the cost of additional computation. While other text- and graph-based generative models tend to struggle with large molecular graphs due to the long autoregression chains needed to produce them, fragment-based graph generative models require autoregression lengths on the order of the number of fragments that comprise a molecule. This can be significant when the fragments themselves contain many atoms.

However, some issues persist due to general difficulties with autoregressive graph generation. Unlike text-based models, in which there is less ambiguity in the autoregression order (e.g., tokens are typically decoded in left-to-right order), graphs have no such canonical node order, which presents challenges for graph-based autoencoders [32, 59]. Furthermore, although they require shorter autoregression chains than their counterparts, existing fragment-based graph generative models nevertheless require autoregression lengths that grow in the overall size of the molecule since autoregressive decoding cannot be effectively parallelized.

While fragment-based graph generative models and SELFIES-based language models each address the issue of chemical validity, there is the separate challenge of synthetic accessibility. Prior work has cast doubt on the synthetic feasibility of compounds proposed by many existing generative models [13], which can limit the practical utility of these models in drug discovery applications if not appropriately addressed. Subsequent work has attempted to improve on these shortcomings by (i) including explicit penalties for synthetic inaccessibility via a scoring function [19], (ii) limiting the model to fragments from known compounds [34, 40, 53], or (iii) inducing bias towards simple and known synthetic pathways [8, 9, 21].

Given the above background, what is needed in the art are scalable approaches for navigating CSLs with query-based random access.

### SUMMARY

The present disclosure addresses the above-identified needs in the art. Systems and methods for querying a combinatorial synthesis library comprising a plurality of compounds and representing a plurality of reaction types, where each reaction type maps to a plurality of reactants, and each reactant maps to a plurality of synthons. The system accepts a query in the form of a single graph into a molecular encoder model, thereby obtaining a query vector. The query vector is inputted into a reaction query generator model thereby obtaining a first reaction type and a first plurality of reactants. A synthon is determined for each reactant by inputting the reactant into a synthon query generator model. A set of synthons is therefore determined, each corresponding to a reactant in the first plurality of reactants. A molecular structure in the combinatorial synthesis library is identified that includes the set of synthons arranged in accordance with a synthesis rule associated with the first reaction type.

In one embodiment the molecular encoder model is a graph-based generative model that exploits the structure of CSLs to provide efficient navigation of the relevant chemical space. The model learns a hierarchy of keys over the components of the library and uses these keys to process queries for retrieval. The encoder processes the molecular graph and returns as output a query vector, which a decoder uses to retrieve the molecule from the CSL through an efficient sequence of query-key comparisons that utilizes the hierarchical construction of CSLs, requiring minimal autoregression and admitting efficient parallelization. The graph-based generative model in such embodiments acts as a “neural database,” providing random access to ultra-large, non-enumerable compound libraries. As such, the model provides valid and cost-effectively accessible molecules. Moreover, the model overcomes challenges with long autoregressive chains in compound generation, improving scalability to large molecular graphs. Further still, the model reduces the number of parameters ten-fold, relative to comparable methods, and offers considerable improvements in computational complexity for searching through CSLs.

System and methods for querying a combinatorial synthesis library are provided. The combinatorial synthesis library comprises a plurality of compounds and represents a plurality of reaction types. Each respective reaction type in the plurality of reaction types has a corresponding mapping to a corresponding plurality of reactants. Each respective reactant in each corresponding plurality of reactants has a corresponding mapping to a corresponding plurality of synthons.

In some such embodiments, the plurality of reaction types comprises or more reaction types and the combinatorial synthesis library comprises or more compounds for each reaction type in the plurality of reaction types.

A molecular query is inputted into a molecular encoder model. The query is graph-based. The molecular encoder model comprises a message passing neural network comprising a plurality of message passing layers that collectively comprise a first plurality of parameters. The inputting of the graph-based query into the molecular encoder model results in a query vector, as output of the molecular encoder model, by application of the first plurality of parameters to the graph-based query

In some such embodiments, the graph-based query is a single graph that comprises a plurality of nodes and a plurality of edges, where each node in the plurality of nodes is connected by at least one edge in the plurality of edges to another node in the plurality of nodes.

In some such embodiments, each respective node in the plurality of nodes is associated with (i) a corresponding element type in a plurality of element types, (ii) a node degree in a plurality of node degrees, (iii) a hybridization in a plurality of hybridizations, (iv) a number of bonded hydrogens, (v) a formal charge from among a set of formal charges, and (vi) a binary indication of aromaticity.

In some such embodiments, each respective bond in the plurality of bonds is associated with (i) a bond type, (ii) a binary indication of conjugation, (iii) a binary of indication of whether or not the respective bond is in a ring, and (iv) an indication of stereochemistry.

In some such embodiments, the graph, which can be an arbitrary graph, represents a single molecular compound, where such single molecular compound is present in the combinatorial synthesis library.

In some such embodiments, the graph represents a weighted composite of a first graph of a first molecular compound and a second graph of a second molecular compound.

In some such embodiments, the graph represents a weighted composite of a plurality of graphs of a second plurality of compounds, where the second plurality of compounds have a common property.

In some such embodiments, the common property is a Tanimoto distance less than a threshold value to each other compound it the second plurality of compounds.

In some such embodiments, the common property is a binding coefficient to macromolecular target that is less than a threshold value.

The query vector is inputted into a reaction query generator model comprising a second plurality of parameters. This results in an output from the reaction query generator model 116 of a first reaction type in the plurality of reaction types by application of the second plurality of parameters to the query vector.

In some such embodiments, the reaction query generator model is a two-layer perceptron with intermediate ReLU activation.

In some such embodiments, the output from the reaction query generator model is used to identify a first reaction key type in a plurality of reaction key types through a first query key lookup, where each reaction key type in the plurality of reaction key types represents a synthetic reaction that can be used to synthesize one or more compounds in the combinatorial synthesis library.

A corresponding synthon is determined for each respective reactant in a first plurality of reactants corresponding to the first reaction type from among the corresponding plurality of synthons mapped to the respective reactant by inputting the respective reactant 86 into a synthon query generator model comprising a third plurality of parameters thereby obtaining, as output from the synthon query generator model, the corresponding synthon by application of the third plurality of parameters to the respective reactant. This results in a set of synthons, each synthon in the set of synthons corresponding to a reactant in the first plurality of reactants.

In some such embodiments, the synthon query generator model is a two-layer perceptron with intermediate ReLU activation.

In some such embodiments, the output from the synthon query generator model is used to identify a synthon key for the corresponding synthon through a second query key lookup.

In some such embodiments, the first plurality of parameters comprises 100,000 parameters, the second plurality of parameters comprises 5,000 parameters, and the third plurality of parameters comprises 5,000 parameters.

In some such embodiments, the first plurality of reactants comprises three or more reactants and the corresponding mapping for the corresponding plurality of synthons for a reactant in the three or more reactants comprises ten or more synthons.

A molecular structure (compound) is identified in the combinatorial synthesis library that includes the set of synthons, for example, arranged in accordance with a synthesis rule associated with the first reaction type.

In some such embodiments, the plurality of compounds in the combinatorial synthesis library comprises a billion or more compounds and the molecular structure that is identified is any one of the billion or more compounds satisfying the query.

In some such embodiments, the plurality of compounds in the combinatorial synthesis library comprises a trillion compounds or more compounds and the molecular structure that is identified is any one of the trillion or more compounds satisfying the query.

### BRIEF DESCRIPTION OF THE FIGURES

In the drawings, embodiments of the systems and methods of the present disclosure are illustrated by way of example. It is to be expressly understood that the description and drawings are only for the purpose of illustration and as an aid to understanding and are not intended as a definition of the limits of the systems and methods of the present disclosure.

FIGS. 1A and 1B illustrate a computer system, in accordance with some embodiments of the present disclosure.

FIGS. 2A, 2B, 2C, and 2D illustrate methods for querying a combinatorial synthesis library in which dashed boxes represent optional elements, in accordance with some embodiments of the present disclosure

FIGS. 3A, 3B, 3C, 3D, 3E, 3F, 3G, and 3H collectively illustrate an overview of systems and methods for screening compound libraries, in accordance with some embodiments of the present disclosure. FIG. 3A: Combinatorial synthesis libraries are comprised of a potentially large number of tables that describe multi-component reactions. An example two-component reaction is illustrated with four synthons per group, resulting in a sixteen product chemical space. Libraries of such construction can describe massive swaths of accessible chemical space via combinatorial explosion, thereby greatly accelerating early-stage drug discovery. FIG. 3B: An example efficient graph generative model is developed and utilized in the navigation of ultra-large combinatorial synthesis libraries. At a high level, the example model first encodes a molecule (represented as a graph) via a GNN encoder. Given the molecular representation, the decoder generates queries into a synthon-based library for retrieval. The decoder is structured in such a way to make retrieval fast and efficient. FIG. 3C: The library encoder begins by learning a representation for each synthon (e.g., 88-1, 88-2, 88-3, 88-4), represented as a molecular graph, via a synthon encoder (GNN). The synthon representations are used to generate keys for each synthon via a multi-layer perceptron (MLP). FIG. 3D: Moving up the hierarchy, group representations are learned via a set neural network operating on the representations of the synthons belonging to each group. FIG. 3E: Similarly, reaction representations are learned via a set neural network operating on the representations of the constituent groups. Reaction keys are also generated from the reaction representations via an MLP. FIG. 3F: The molecular encoder is a graph neural network that takes a molecular graph as input and produces a molecular key as output, which is used to query a synthon-based library. FIG. 3G: To reconstruct the input molecule, a reaction query is first generated from the molecular key which is then compared against the reaction keys for sampling the reaction type. FIG. 3H: Next, a synthon query is generated for each group independently (e.g., no autoregression) using the molecular key, reaction representation, and group representation, which is compared against the synthon keys for sampling the synthons (e.g., 98-1, 98-2, 98-3, 98-4, 98-5, 98-6, 98-7, and 98-8).

FIGS. 4A and 4B collectively illustrate interpolations in latent space, in accordance with an embodiment of the present disclosure. FIG. 4A: Moving left to right in raster order, linear interpolation is performed from the starting molecule to the target molecule. Below each molecule are its Tanimoto similarities with the starting and target molecule, respectively. FIG. 4B: Two random directions are sampled in the latent space around a query compound and the decoded molecules are visualized as being spaced evenly on the resulting two-dimensional plane.

FIGS. 5A and 5B collectively illustrate autoencoding as analog, in accordance with an embodiment of the present disclosure. Each query molecule (top left indicated by box) is autoencoded and stochastically decoded in accordance with the systems and methods of the present disclosure fifteen times. Below each molecule is its Tanimoto similarity with the query molecule. FIG. 5A: Autoencoding a molecule contained in the library. FIG. 5B: Autoencoding a molecule not contained in the library.

FIG. 6 illustrates synthons in accordance with the prior art.

FIG. 7 illustrates how, as the number of distinct reactions present in training increases, the in-library proportion for generated samples from RationaleRL quickly decreases, dropping to below 50% after just 50 reactions are included.

FIG. 8 illustrates test-time distribution shift as a function of library size, in accordance with some embodiments of the present disclosure.

FIG. 9 illustrates an ex-post density estimation procedure, in accordance with some embodiments of the present disclosure.

FIG. 10 illustrates a list of United States Federal Drug Administration novel drug approvals in 2021 used in analog comparison, in accordance with some embodiments of the present disclosure.

FIG. 11 illustrates a comparison of CSLVAE to Arthor in top-1 analogue search in accordance with some embodiments of the present disclosure.

Like reference numerals refer to corresponding parts throughout the several views of the drawings.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

Unless defined otherwise, all technical and scientific terms used herein have the same meaning as is commonly understood by one of skill in the art to which this disclosure belongs. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” include the compounds described herein, and any pharmaceutically acceptable analogs, derivatives, salts, solvates, hydrates, cocrystals, or prodrugs thereof. The terms “active pharmaceutical ingredient” and “drug” may also include those compounds described herein and any pharmaceutically acceptable analogs, derivatives, salts, solvates, hydrates, cocrystals, or prodrugs thereof that bind a target molecule.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells. The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs disclosed herein, can also be incorporated into the described compositions and methods.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, or from 0% to 10%, or from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

As used interchangeably herein, the term “classifier” or “model” refers to a machine learning model or algorithm.

In some embodiments, a “model” is supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a classifier is a multinomial classifier algorithm. In some embodiments, a model is a 2-stage stochastic gradient descent (SGD) model. In some embodiments, a model is a deep neural network (e.g., a deep-and-wide sample-level classifier).

Neural networks. In some embodiments, a model is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) can be a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xᵢ, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in accordance with the present disclosure. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used in accordance with the present disclosure.

For instance, a deep neural network classifier comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network classifier. In some embodiments, at least 100 parameters, at least 1000 parameters, at least 2000 parameters or at least 5000 parameters are associated with the deep neural network classifier. As such, deep neural network classifiers require a computer to be used because they cannot be mentally solved. In other words, given an input to the classifier, the classifier output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as classifiers are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as classifiers are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as classifiers are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×106, n≥5×10⁶, or n≥1×10⁷. As such, the algorithms, models, regressors, and/or classifiers of the present disclosure cannot be mentally performed. In some embodiments n is between 10,000 and 1×107, between 100,000 and 5×106, or between 500,000 and 1×106. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

For the avoidance of doubt, it is intended herein that particular features (for example integers, characteristics, values, uses, diseases, formulae, compounds or groups) described in conjunction with a particular aspect, embodiment or example of the disclosure are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All embodiments of the invention can, in the alternative, be more specifically defined by any of the transitional terms “comprising,” “consisting essentially of,” and “consisting of.”

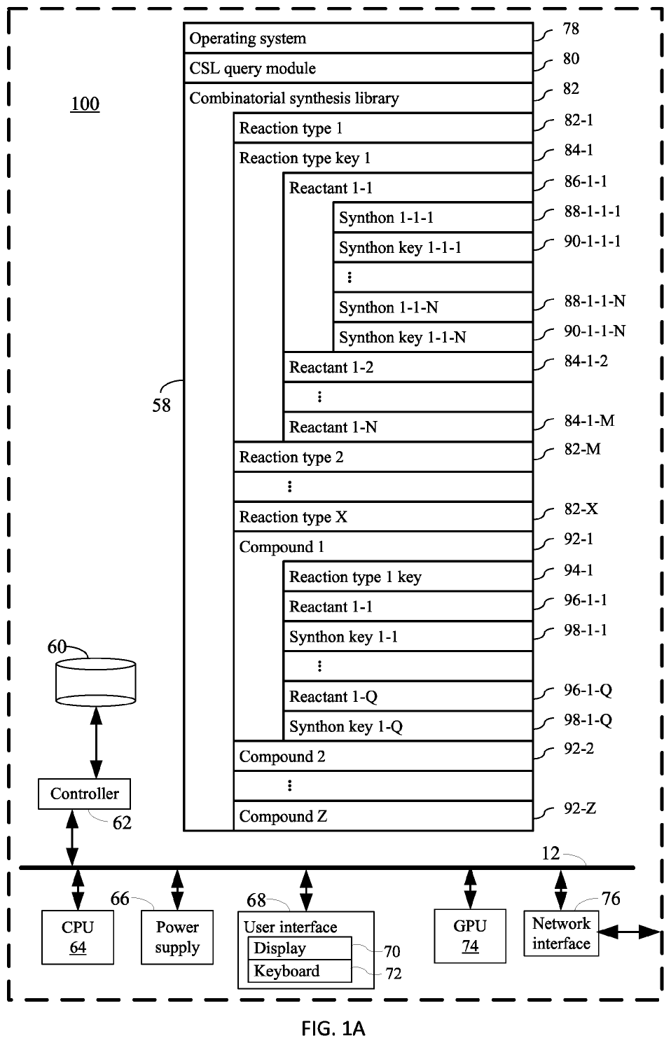

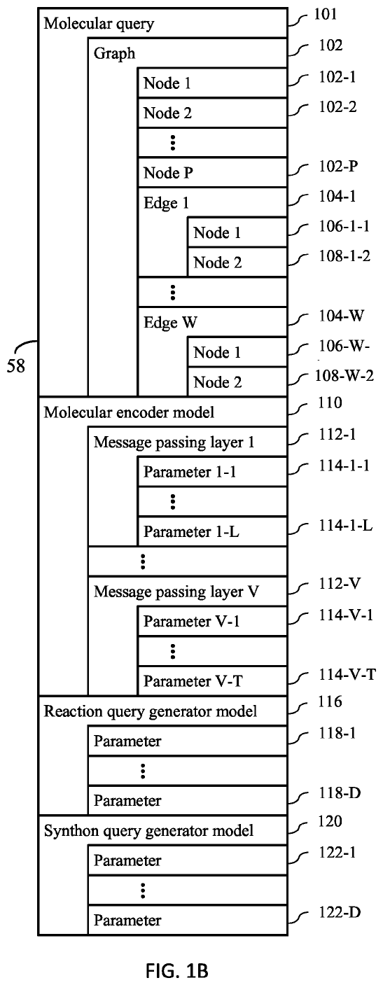

FIGS. 1A and 1B illustrate a computer system 100 for querying a combinatorial synthesis library comprising. Referring to FIGS. 1A and 1B, in typical embodiments, computer system 100 comprises one or more computers. For purposes of illustration in FIGS. 1A and 1B, the computer system 100 is represented as a single computer that includes all of the functionality of the disclosed computer system 100. However, the present disclosure is not so limited. The functionality of the computer system 100 may be spread across any number of networked computers and/or reside on each of several networked computers and/or virtual machines. One of skill in the art will appreciate that a wide array of different computer topologies are possible for the computer system 100 and all such topologies are within the scope of the present disclosure.

Turning to FIGS. 1A and 1B with the foregoing in mind, the computer system 100 comprises one or more central processing units (CPUs) 64, one or more graphic processing units (GPUs) 74, a network or other communications interface 76, a user interface 68 (e.g., including an optional display 70 and optional keyboard 72 or other form of input device), a memory 58 (e.g., random access memory, persistent memory, or combination thereof), one or more magnetic disk storage and/or persistent devices 60 optionally accessed by one or more controllers 62, one or more communication busses 12 for interconnecting the aforementioned components, and a power supply 66 for powering the aforementioned components. To the extent that components of memory 58 are not persistent, data in memory 58 can be seamlessly shared with non-volatile memory 60 using known computing techniques such as caching. Memory 60 can include mass storage that is remotely located with respect to the central processing unit(s) 64. In other words, some data stored in memory 58 and/or memory 60 may in fact be hosted on computers that are external to computer system 100 but that can be electronically accessed by the computer system 100 over an Internet, intranet, or other form of network or electronic cable using network interface 76. In some embodiments, the computer system 100 makes use of models that are run from the memory associated with one or more graphical processing units 74 in order to improve the speed and performance of the system. In some alternative embodiments, the computer system 100 makes use of models that are run from memory 58 rather than memory associated with a graphical processing unit.

The memory 58 of the computer system 100 stores:
an optional operating system 78 that includes procedures for handling various basic system services;
a combinatorial synthesis library query model 80 for querying a combinatorial synthesis library;
a combinatorial synthesis library 82 comprising a plurality of compounds 92, the combinatorial synthesis library representing a plurality of reaction types 82, each such reaction type indexed by a reaction type key 84 and having a corresponding mapping to a corresponding plurality of reactants, each respective reactant 86 in each corresponding plurality of reactants having a corresponding mapping to a corresponding plurality of synthons 88, each such synthon indexed by a synthon key 90, so that each respective compound 92 is indexed by reaction type key 94 which, in turn, can be used to determine reactants 96 and synthon keys 98 for the respective compound;
a molecular query 101 that is used to query the combinatorial synthesis library 82, where the molecular query is in the form of a graph 102 comprising a plurality of nodes 102 and a plurality of edges 104, where each node in the plurality of nodes represents an atom and each node is connected by at least one edge, in the plurality of edges, to another node in the plurality of nodes, the edge representing a covalent bond between the two nodes;
a molecular encoder model 110 optionally comprising a message passing neural network comprising a plurality of message passing layers 112 that collectively comprise a first plurality of parameters 114, the molecular encoder model, in response to receiving a molecular query 101 in the form of a graph 102, outputting a query vector by application of the plurality of parameters 114 to the graph 102;
a reaction query generator model 116 comprising a second plurality of parameters 118, the reaction query generator model 116, in response to receiving the query vector, providing as output from the reaction query generator model 116, a first reaction type 82-1 in the plurality of reaction types 82 by application of the second plurality of parameters 118 to the query vector; and
a synthon query generator model 120 comprising a third plurality of parameters 122, the synthon query generator model 120, in response to receiving an identify of a reactant, providing, as output from the synthon query generator model 120, the corresponding synthon 88 by application of the third plurality of parameters 122 to the respective reactant.

In some implementations, one or more of the above identified data elements or modules of the computer system 100 are stored in one or more of the previously mentioned memory devices and correspond to a set of instructions for performing a function described above. The above identified data, modules or programs (e.g., sets of instructions) need not be implemented as separate software programs, procedures or modules, and thus various subsets of these modules may be combined or otherwise re-arranged in various implementations. In some implementations, the memory 60 (and optionally memory 58) optionally stores a subset of the modules and data structures identified above. Furthermore, in some embodiments the memory 60 (and optionally memory 58) stores additional modules and data structures not described above.

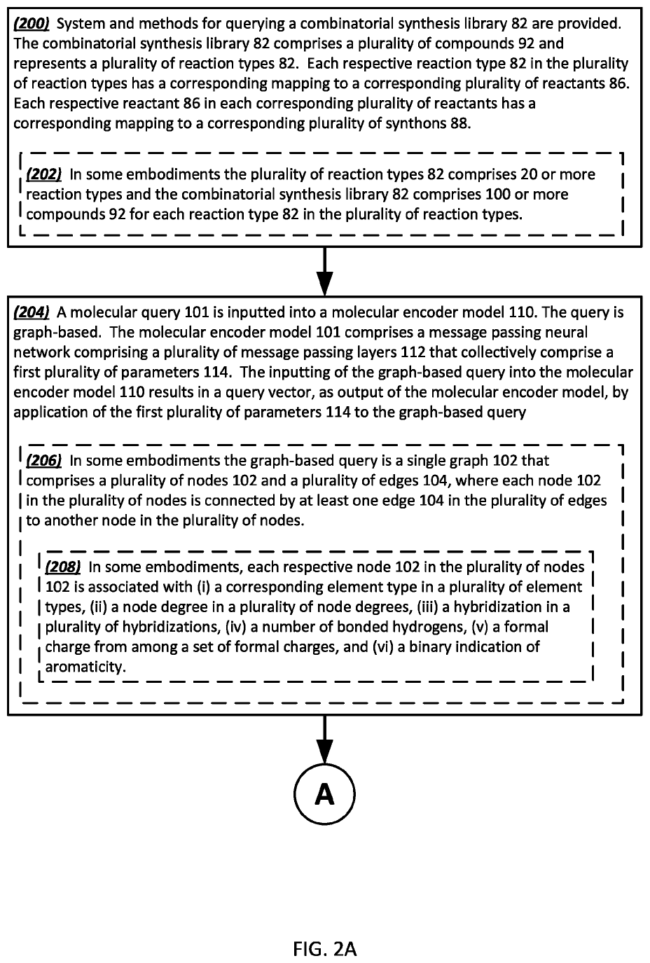

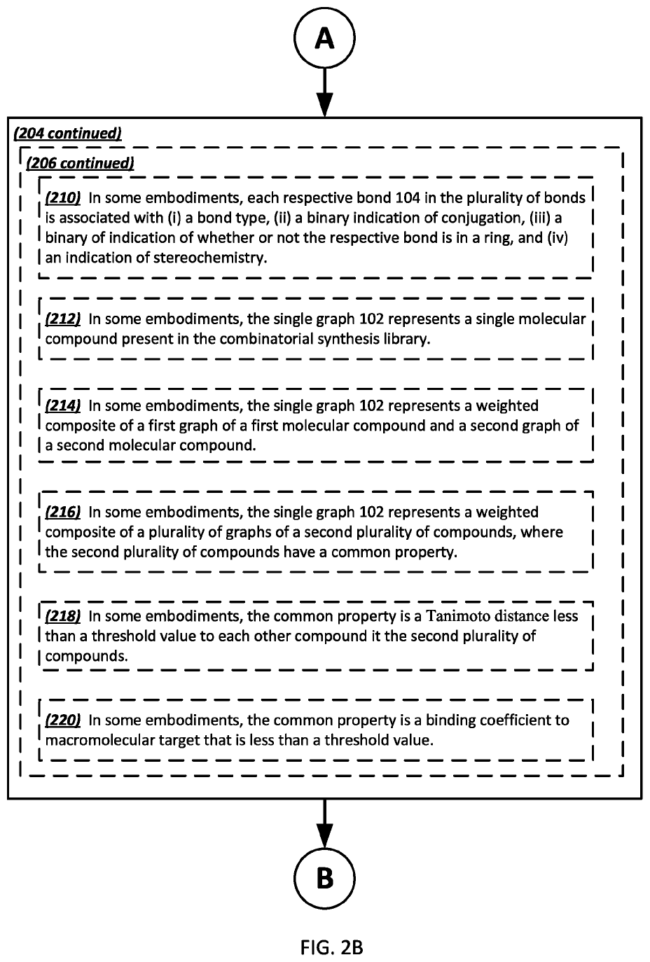

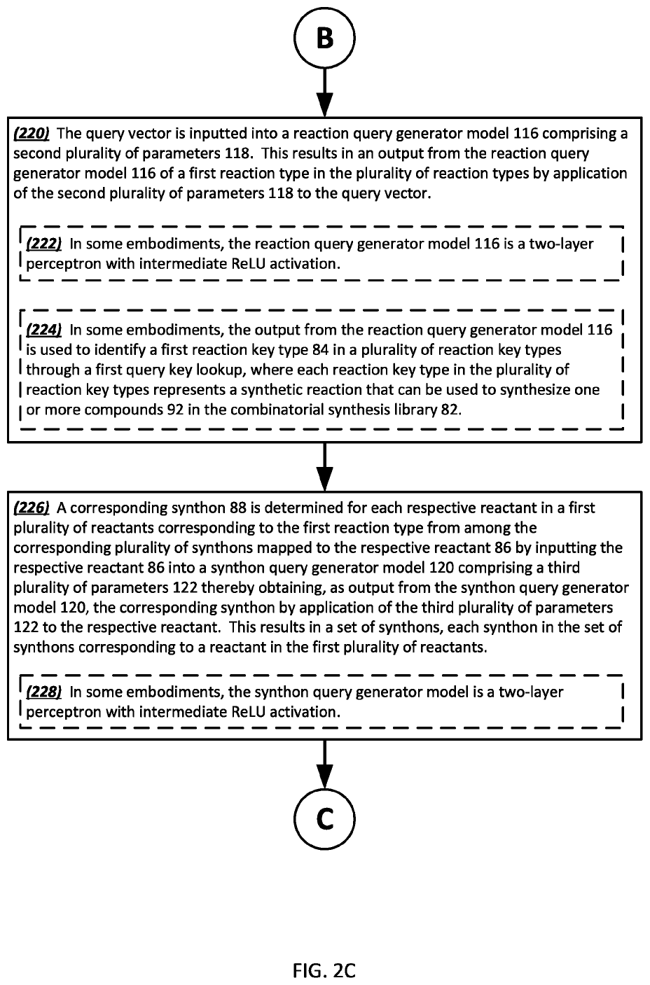

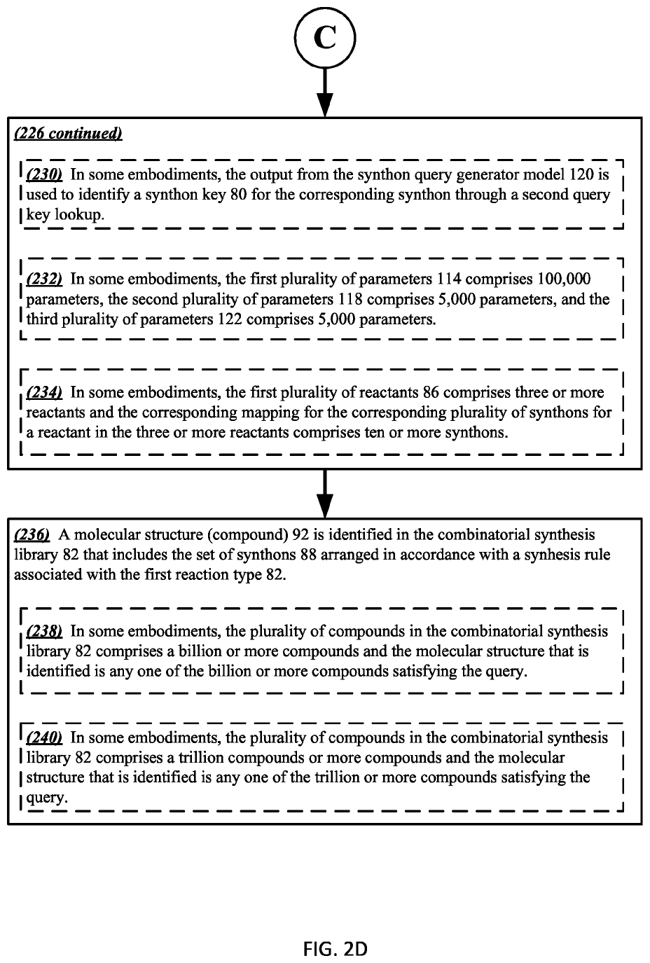

Now that a system for querying a combinatorial synthesis library has been disclosed, methods for performing such queries is detailed with reference to FIG. 2 and discussed below.

Referring to block 200, in some embodiments, system and methods for querying a combinatorial synthesis library 82 are provided. The combinatorial synthesis library 82 comprises a plurality of compounds 92 and represents a plurality of reaction types 82. Each respective reaction type 82 in the plurality of reaction types has a corresponding mapping to a corresponding plurality of reactants 86. Each respective reactant 86 in each corresponding plurality of reactants has a corresponding mapping to a corresponding plurality of synthons 88.

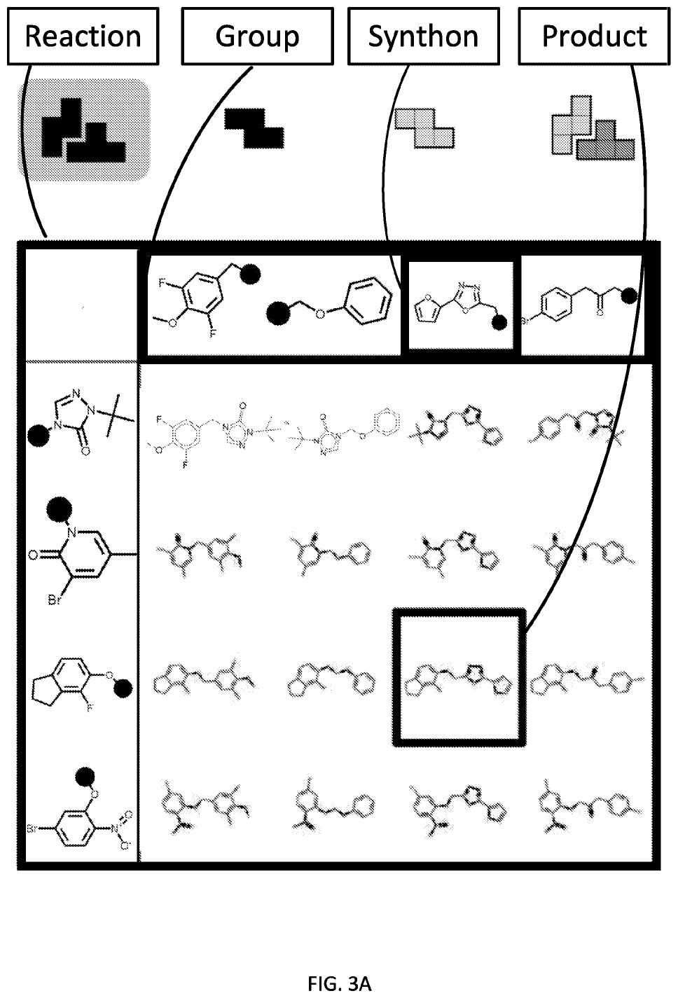

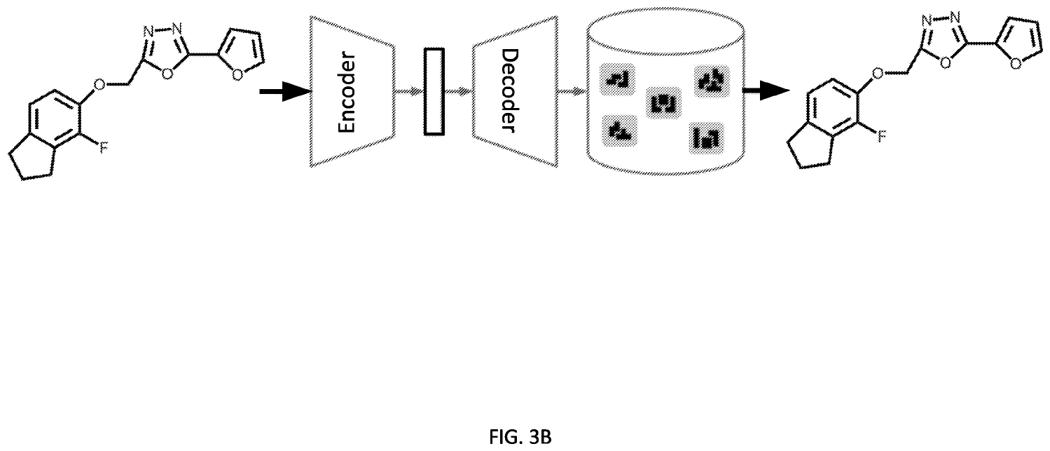

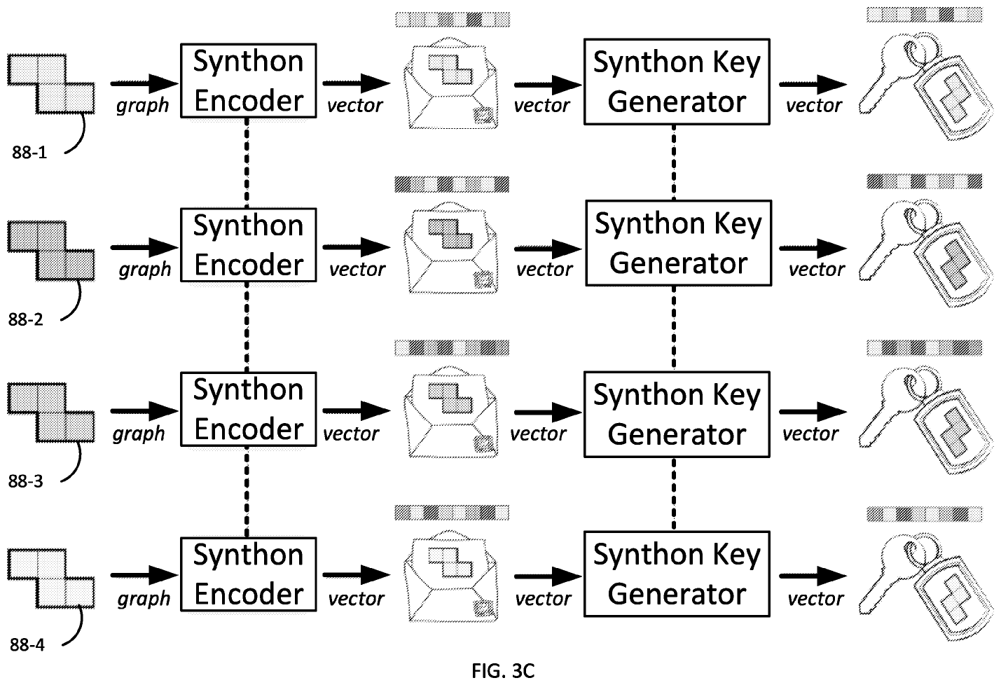

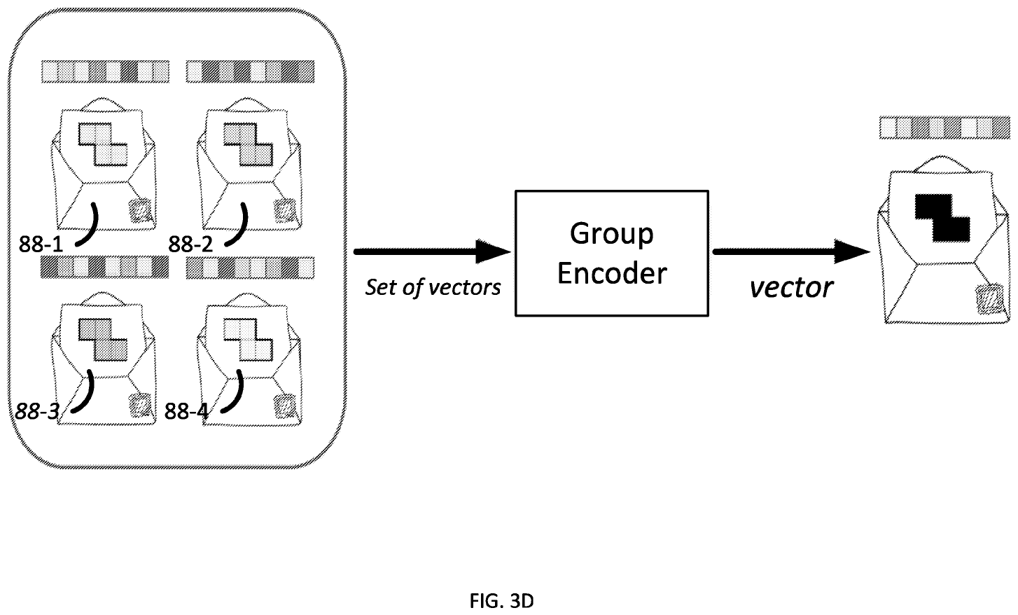

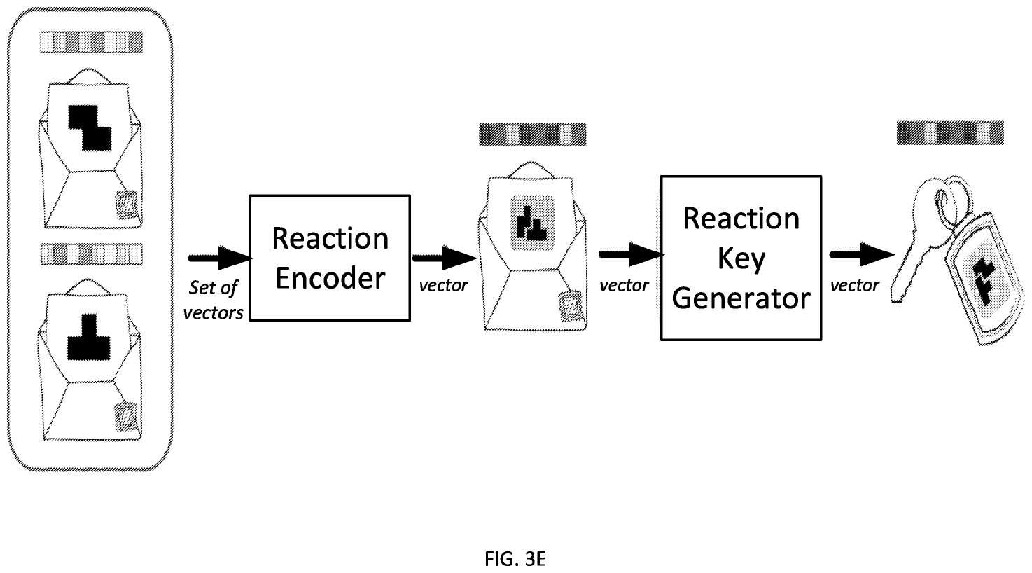

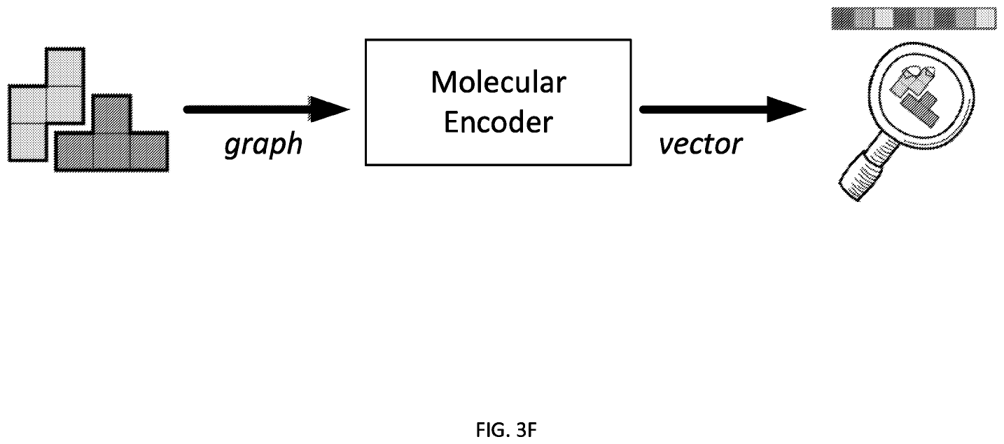

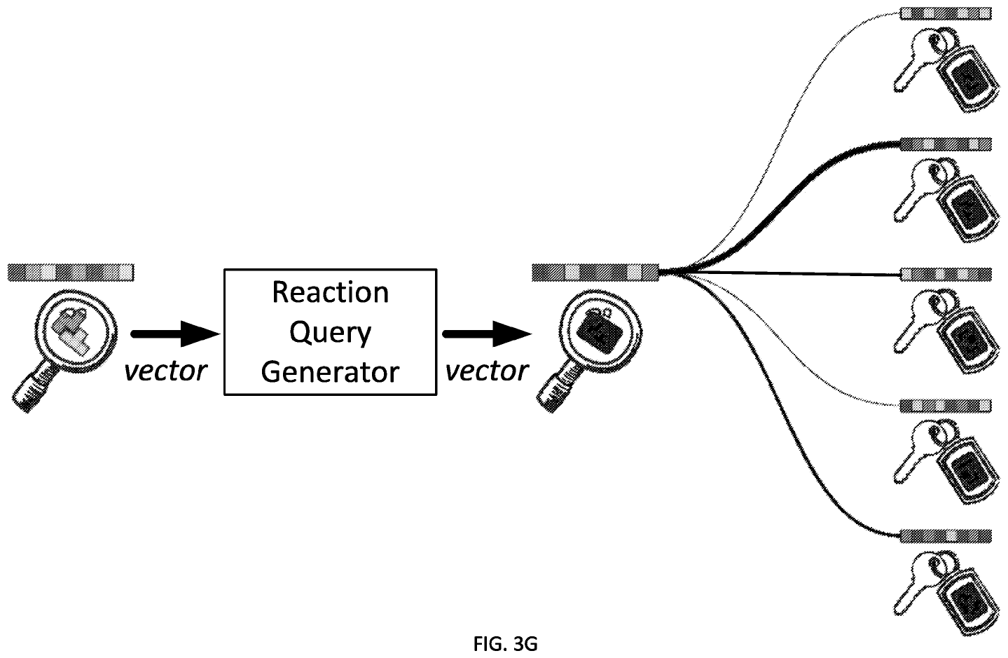

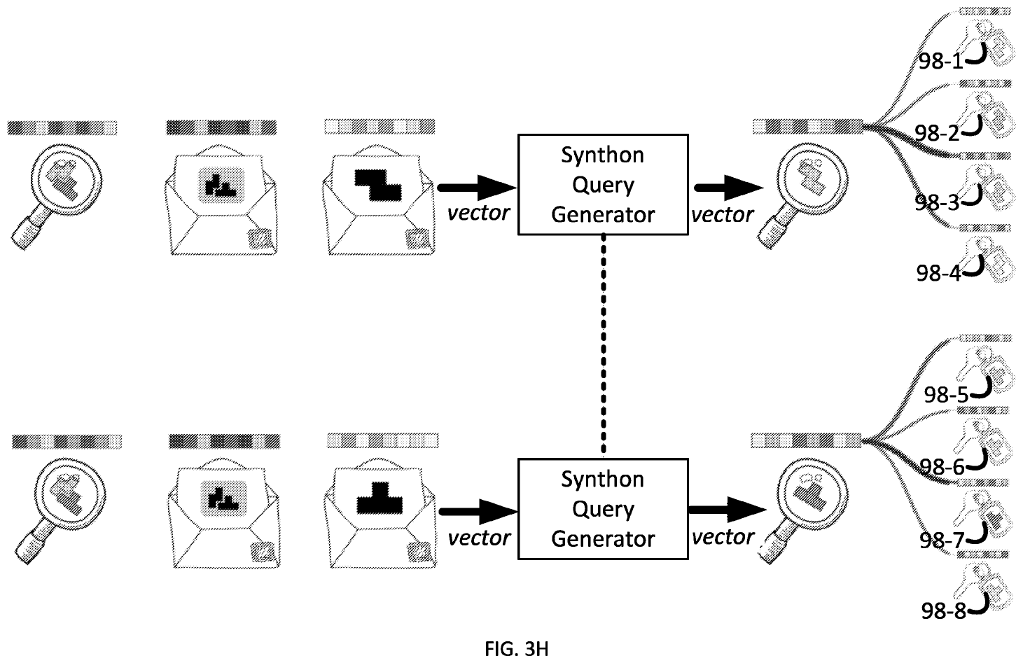

One aspect of the present disclosure focuses on combinatorial synthesis chemical libraries (CSLs), which can be used to represent vast swaths of accessible chemical space (e.g., more than 10⁸, 10⁹, 10¹⁰, or 10¹¹ compounds) via combination of a smaller set of readily available building blocks and a set of pre-described and generally successful multi-component synthesis rules. An illustration of such constructions is provided in FIG. 3A. In some embodiments, CSLs are composed of a set of multi-dimensional synthesis tables. In some such embodiments, each synthesis table describes a multi-component chemical reaction, which can be denoted by index/E T (e.g., a natural number).

Following [44], the term “R-group,” or group for short, is used to refer to a placeholder group in a chemical reaction, and is denoted by r E R. Multiple groups can be combined together in a (chemically valid) reaction, which is denoted by t∈T. We let ψ: T→P(R) be the function that returns the set of R-groups ψ(t)⊂R required for reaction t.

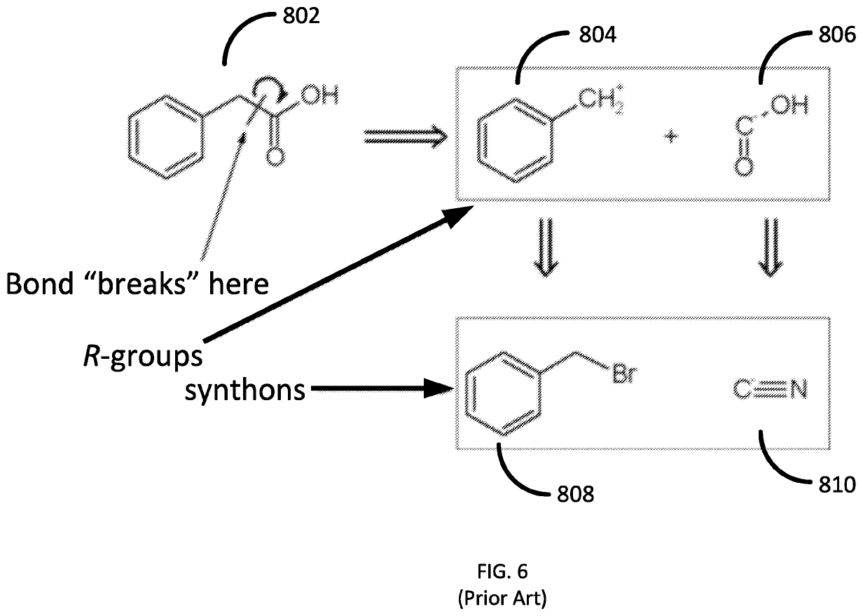

Each R-group is spanned by a possibly large number of molecular building blocks, called synthons, which can be utilized in the corresponding reaction. A synthon is denoted by s∈S and can be represented with a molecular graph, [symbol] ₛ∈ [symbol] ₛ. It is noted that a synthon can belong to multiple R-groups. A target molecule is represented by R-groups that represent a potential starting reagent in the synthesis of that target molecule. For example, in FIG. 6, compound 602 comprises two R-groups, the electrophilic “PhCH²⁺” R-group 604 and the nucleophilic “COOH⁻” R-group 606. Both R-groups do not exist per se; a synthetic equivalent (synthon) corresponding to each R-group, is reacted to produce the desired R-groups and thus the overall compound. For example, in the illustration provided in FIG. 6, the cyanide anion 610 is one possible synthon for the “COOH⁻” R-group 606, while benzyl bromide 608 is one possible synthon for the “PhCH²⁺” R-group 604.

For convenience, in some embodiments, the notation σ: R→ [symbol] (S) is used to represent the function the returns the set of synthons σ(r)⊂S belonging to a particular R-group r.

A product x∈X is a molecule that is synthesized according to a multi-component reaction with R-groups

$$ψ ( t ) = ( {r}_{t}^{( 1 )} , … {r}_{t}^{( {k}_{t} )} )$$

and a corresponding synthon tuple

$$u = ( {s}_{u}^{( 1 )} , … {s}_{u}^{( {k}_{t} )} ) ,$$

where

$${s}_{u}^{( i )} ∈ σ ( {r}_{t}^{( i )} )$$

for all i=1, . . . , kₜ (kₜ is between 2 and 4 in some embodiments). Let f be defined as the synthesis rule that generates a compound x from a reaction and synthon tuple pair (t, u), e.g., x: =f(t, u). In short, as a simple analogy, one can think of a synthesis rule t as specifying an equation of kₜ terms, where each term is an R-group, and their associated synthons correspond to the allowed values for the corresponding term.

Hence, a synthon-based library [symbol] =(T, R, S, f, ψ, σ) is fully characterized by its reactions T, R-groups R, and synthons S, together with the synthesis rule f, reaction to R-groups mapping ψ, and R-group to synthons mapping σ. Due to the combinatorial nature of such constructions, they can be used to build libraries that span vast regions of readily accessible chemical space from which compounds can be acquired (i) with high probability, (ii) at reasonable cost, and (iii) with low lead times.

Using the language of probability, one can describe a distribution over X induced by D via the following factorization:

$$p ( x \vert 𝒟 ) = {\sum }_{t ∈ T} {\sum }_{u ∈ {U}_{t}} p ( t \vert 𝒟 ) p ( u \vert t , 𝒟 ) , {p}_{f} ( x \vert t , u ) , \qquad ( 1 )$$

where

$${U}_{t} = σ ( {r}_{t}^{( 1 )} ) \times … \times σ ( {r}_{t}^{( {k}_{t} )} )$$

is the set of all eligible synthon chains for reaction t. This factorization describes the generative process in which one first samples a reaction t˜p(t| [symbol] )∝|Uₜ|, then samples a valid synthon tuple u˜p(u|t, [symbol] )=|Uₜ|⁼¹ comprised of synthons from the respective groups in t, and joins these together via synthesis to form a product x (via the deterministic rule f).

As written, all valid (t, u) pairs in [symbol] are equally probable under p. Note that if every product in [symbol] can be reached according to just a single synthesis route, then p(x| [symbol] ) is a uniform distribution over the part of X accessible by [symbol] .

Referring to block 202, in some embodiments the plurality of reaction types 82 comprises 20 or more reaction types and the combinatorial synthesis library 82 comprises 100 or more compounds 92 for each reaction type 82 in the plurality of reaction types. In some embodiments the plurality of reaction types 82 comprises 20, 30, 40, 50, 60, 70, 80, 90, 100, 500, 1000, 2000, 3000, 5000, 10,000 or more reaction types and the combinatorial synthesis library 82 comprises 100, 200, 300, 400, 500, 1000, or 10,000 or more compounds 92 for each reaction type 82 in the plurality of reaction types.

Referring to block 204, a molecular query 101 is inputted into a molecular encoder model 110. The query is graph-based. In some embodiments, the molecular encoder model 101 comprises a message passing neural network comprising a plurality of message passing layers 112 that collectively comprise a first plurality of parameters 114. The inputting of the graph-based query into the molecular encoder model 110 results in a query vector, as output of the molecular encoder model, by application of the first plurality of parameters 114 to the graph-based query.

In some embodiments, the molecular encoder model 101 (MolecularEncoder) [symbol] _{X}→ [symbol] ^{d}^{ₓ} takes as input a molecular graph [symbol] _{X}∈ [symbol] _{X} and returns a d_{X}-dimensional feature representation:

$$z = MolecularEncod er ( {𝒢}_{x} ) \qquad ( 10 )$$

In some implementations, the MolecularEncoder (molecular encoder model 101) is a graph neural network with a variational linear layer stacked on top of the graph readout, which produces a sample z˜q(z|x) for a given input graph [symbol] ₓ. As depicted in FIGS. 3A-H, the representation z is interpreted as a query induced by x into the library [symbol] .

Note that, in some embodiments, molecular encoder model 110 takes as input a molecular graph and is therefore capable of producing queries for compounds that are not in the library [symbol] . This is useful in some embodiments, for finding analogs by catalog—that is, compounds that can be purchased from a catalog and are chemical analogs of a query molecule.

Referring to block 206, in some embodiments the graph-based query is a single graph 102 that comprises a plurality of nodes 102 and a plurality of edges 104, where each node 102 in the plurality of nodes is connected by at least one edge 104 in the plurality of edges to another node in the plurality of nodes. In some embodiments, the single graph 102 represents a single molecular compound and each node in the plurality of nodes represents an atom of the single molecular compound and each edge in the plurality of edges represents a covalent bond between two atoms in the single molecular compound. In some embodiments, the plurality of nodes represents each non-hydrogen atom of a molecular compound. In some embodiments, the plurality of nodes represents each atom of a molecular compound. In some embodiments, the plurality of edges represents each covalent bond of a molecular compound.

In some such embodiments, each respective node in the plurality of nodes is associated with any 1, 2, 3, 4 or more of the following characteristics: (i) a corresponding element type in a plurality of element types, (ii) a node degree in a plurality of node degrees, (iii) a hybridization in a plurality of hybridizations, (iv) a number of bonded hydrogens, (v) a formal charge from among a set of formal charges, and (vi) a binary indication of aromaticity.

Referring to block 208, in some embodiments, each respective node 102 in the plurality of nodes 102 is associated with (i) a corresponding element type in a plurality of element types, (ii) a node degree in a plurality of node degrees, (iii) a hybridization in a plurality of hybridizations, (iv) a number of bonded hydrogens, (v) a formal charge from among a set of formal charges, and (vi) a binary indication of aromaticity.

Referring to block 210, in some embodiments, each respective bond 104 in the plurality of bonds is associated with (i) a bond type, (ii) a binary indication of conjugation, (iii) a binary of indication of whether or not the respective bond is in a ring, and (iv) an indication of stereochemistry.

In some embodiments, atoms and bonds of a molecular compound are represented in a graph as a set of binary features. In some embodiments these binary features are described in Tables 1 and 2, respectively. In one example implementation, the node and edge dimensions are set to 64 each. Thus, in this example, the atom embeddings consist of 50×64=3,200 parameters, and the bond embeddings consist of 12× 64=768 parameters.

TABLE 1
Atom feature types.

| | | Number of |
|---|---|---|
| Feature | Choices | choices |
| Element type | *, B, Br, C, Cl, F, Fe, I, | 15 |
| | N, O, P, S, Se, Si, Sn | |
| Node degree | 1, 2, 3, 4, 5, 6, ≥7 | 7 |
| Hybridization | S, SP, SP2, SP3, SP3D, SP3D2, | 7 |
| | Unspecified | |
| Chirality | CW, CCW, Other, Unspecified | 4 |
| Bonded hydrogens | 0, 1, 2, 3, 4, 5, 6, ≥7 | 8 |
| Formal charge | ≤−3, −2, −1, 0, 1, 2, ≥3 | 7 |
| Aromatic | False, True | 2 |
| | | 50 |

TABLE 2
Bond feature types.

| | | Number of |
|---|---|---|
| Feature | Choices | choices |
| Bond type | Single, Double, | 4 |
| | Triple, Aromatic | |
| Conjugated | False, True | 2 |
| In a ring | False, True | 2 |
| Stereochemistry | One, Z, E, Any | 4 |
| | | 12 |

In some embodiments, the single graph represents a query molecular compound as a set of atom features and a set of bond features. For instance, in some embodiments, the single graph represents a query molecular compound as a set of atom features and a set of bond features of Tables 1 and 2, respectively. In some such embodiments the set of atom features comprises element type, node degree, hybridization, chirality, bonded hydrogens, formal charge, aromaticity, and the set of bond features comprises bond time, conjugated, in a ring, and stereochemistry as set forth in Tables 1 and 2. In some such embodiments, each non-hydrogen atom in the query molecular compound is represented by 500, 1000, 1500, 2000, 2500, 3000 or more than 3000 parameters in the set of atom features and each covalent bond in the molecular compound is represented by 100, 200, 300, 400, 500, 600, 700, or more than 700 more parameters in the set of bond features.

Referring to block 212, in some embodiments, the single graph 102 represents a single molecular compound present in the combinatorial synthesis library. In some embodiments, the single graph 102 represents a single molecular compound that is not present in the combinatorial synthesis library.

Referring to block 214, in some embodiments, the single graph 102 represents a weighted composite of a first graph of a first molecular compound and a second graph of a second molecular compound. In some embodiments, the single graph 102 represents a composite of two or more molecular compounds.

Referring to block 216, in some embodiments, the single graph 102 represents a weighted composite of a plurality of graphs of a second plurality of compounds, where the second plurality of compounds have a common property. Referring to block 218, in some embodiments, the common property is a Tanimoto distance less than a threshold value to each other compound it the second plurality of compounds. Referring to block 220, in some embodiments, the common property is a binding coefficient to macromolecular target that is less than a threshold value.

Referring to block 220, the query vector z, (e.g., represented by equation above) is inputted into a reaction query generator model 116 comprising a second plurality of parameters 118. This results in an output from the reaction query generator model 116 of a first reaction type in the plurality of reaction types by application of the second plurality of parameters 118 to the query vector.

In some embodiments in accordance with block 220, given the query MolecularEncoder ( [symbol] ₓ) of block 204 and a library [symbol] , a decoder is tasked with retrieving a molecule responsive to the query from library [symbol] —for instance, in other words, identifying the reaction and synthon tuple that will yield the molecule as a product. As per the previously discussed factorization, in some implementations, decoding proceeds by generating the reaction first.

In some embodiments a reaction query generator model 116 is utilized. In some embodiments the reaction query generator model 116 can be denoted by the operation

$${q}^{T} = ReactionQueryGenerator ( z ) . \qquad ( 11 )$$

In some such embodiments, ReactionQueryGenerator samples [symbol] ^{d}^{_{X}}→ [symbol] ^{k}^{_{T}}, which generates a reaction query q^{T} from the query z to compare against the reaction keys of CSL [symbol] . In some embodiments, the operation q^{T}=ReactionQueryGenerator (z) performed by some embodiments of reaction query generator model 116 is mathematically equivalent to:

$${\{ p ( {t}_{j} \vert z , 𝒟 ) \}}_{j = 1}^{\vert T \vert} = softmax ( {\{ \frac{{q}^{T} \cdot {k}_{{t}_{j}}^{s}}{\sqrt{{k}_{T}}} \}}_{j = 1}^{\vert T \vert} ) . \qquad ( 12 )$$

thereby defining a probability distribution over reaction types in T. A sampling can be performed according to this probability distribution to arrive at a reaction t˜p(t|z, [symbol] ).

Accordingly, referring to block 224, in some embodiments, the output from the reaction query generator model 116 is used to identify a first reaction key type 84 in a plurality of reaction key types through a first query key lookup, where each reaction key type in the plurality of reaction key types represents a synthetic reaction that can be used to synthesize one or more compounds 92 in the combinatorial synthesis library 82. In some such embodiments, the output from the reaction query generator model 116 is a reaction key type t. In other embodiments, the output from the reaction query generator model 116 is a probability distribution of key types p(t|z, [symbol] ), such as the probability distribution derived from equation (12), from which a particular reaction key type/is obtained by sampling the probability distribution.

Referring to block 222, in some embodiments, the reaction query generator model 116 is a two-layer perceptron (e.g., not counting the input layer and the output layer) with intermediate ReLU activation. In some embodiments, the reaction query generator model 116 is a N-layer perceptron (MLP) where N is a positive integer of 2 or greater (e.g., 2, 3, 4, 5, 6, 7, 8, 9 or 10) and represents the number of hidden layers in the model. In some embodiments, a MLP is a class of feedforward artificial neural network (ANN) comprising at least three layers of nodes: an input layer, one or more hidden layers and an output layer. In such embodiments, except for the input nodes, each node is a neuron that uses a nonlinear activation function. In some embodiments the activation function is rectified linear unit ReLU activation. In some embodiments the activation function is a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof. More disclosure on suitable MLPs that can serve as the reaction query generator model 116 in some embodiments of the present disclosure is found in Vang-mata ed., 2020, Multilayer Perceptrons: Theory and Applications, Nova Science Publishers, Hauppauge, New York, which is hereby incorporated by reference. In some embodiments, the reaction query generator model 116 has the architecture of any of the models disclosed in the definitions section above.

Given the sampled reaction/from block 220, the required R-groups (via) are known and furthermore the synthons eligible for each group (via σ) are known. Accordingly, referring to block 226, in some embodiments, a corresponding synthon 88 is determined for each respective reactant/in the first plurality of reactants corresponding to the first reaction type from among the corresponding plurality of synthons mapped to the respective reactant 86 by inputting the respective reactant 86 into a synthon query generator model 120 comprising a third plurality of parameters 122 thereby obtaining, as output from the synthon query generator model 120, the corresponding synthon by application of the third plurality of parameters 122 to the respective reactant. This results in a set of synthons, each synthon in the set of synthons corresponding to a reactant in the first plurality of reactants. In other words, to decode the synthon tuple for reaction t, a synthon query generator model 120 is implemented. In some embodiments, synthon query generator model 120 implements the operation SynthonQueryGenerator: [symbol] ^{d}^{_{X}}^{+d}^{_{T}}^{++d}^{_{R}}→ [symbol] ^{k}^{_{S}} which is used in some embodiments of the present disclosure to query synthons for each R-group rₜ⁽ⁱ⁾∈ψ(t) as follows:

$${q}_{t , r}^{S} = SynthonQueryGenerator ( \vert z \vert \vert {h}_{t}^{T} \vert \vert {h}_{r}^{R} \vert ) , \qquad ( 13 )$$

$${\{ p ( {s}_{j}^{( i )} \vert z , t , {r}_{t}^{( i )} , 𝒟 ) \}}_{j = 1}^{\vert σ ( {r}_{t}^{( i )} ) \vert} = softmax ( {\{ \frac{{q}_{t , {r}_{t}^{( i )}}^{s} \cdot {k}_{{s}_{j}^{( i )}}^{s}}{\sqrt{{k}_{S}}} \}}_{j = 1}^{\vert σ ( {r}_{t}^{( i )} ) \vert} ) \qquad ( 14 )$$

Referring to block 228, in some embodiments, the synthon query generator model 120 is a two-layer perceptron with intermediate ReLU activation. In some embodiments, the synthon query generator model 120 is a N-layer perceptron (MLP) where N is a positive integer of 2 or greater (e.g., 2, 3, 4, 5, 6, 7, 8, 9 or 10) and represents the number of hidden layers in the model. In some embodiments, a MLP is a class of feedforward artificial neural network (ANN) comprising at least three layers of nodes: an input layer, one or more hidden layers and an output layer. In such embodiments, except for the input nodes, each node is a neuron that uses a nonlinear activation function. In some embodiments the activation function is rectified linear unit ReLU activation. In some embodiments the activation function is a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof. More disclosure on suitable MLPs that can serve as synthon query generator model 120 in some embodiments of the present disclosure is found in Vang-mata ed., 2020, Multilayer Perceptrons: Theory and Applications, Nova Science Publishers, Hauppauge, New York, which is hereby incorporated by reference. In some embodiments, the synthon query generator model 120 has the architecture of any of the models disclosed in the definitions section above.

Referring to block 230, in some embodiments, the output from the synthon query generator model 120 is used to identify a synthon key 80 for the corresponding synthon through a second query key lookup.

Referring to block 232, in some embodiments, the first plurality of parameters 114 (of the molecular encoder model 110) comprises 100,000 parameters, the second plurality of parameters 118 (of the reaction query generator model 116) comprises 5,000 parameters, and the third plurality of parameters 122 (of the synthon query generator model 120) comprises 5,000 parameters. In some embodiments, the first plurality of parameters 114 (of the molecular encoder model 110) comprises 10,000, 50,000, 100,000 or 1×10⁶ parameters, the second plurality of parameters 118 (of the reaction query generator model 116) comprises 1,000, 2,000, 3,000, 4,000, 5,000 or 10,000 parameters, and the third plurality of parameters 122 (of the synthon query generator model 120) comprises 1,000, 2,000, 3,000, 4,000, 5,000 or 10,000 parameters.

Referring to block 234, in some embodiments, the first plurality of reactants 86 comprises three or more reactants and the corresponding mapping for the corresponding plurality of synthons for a reactant in the three or more reactants comprises ten or more synthons. In some embodiments, the first plurality of reactants 86 comprises 2, 3, 4, 5, 6, 7, 8, 9, or 10 or more reactants. In some embodiments the corresponding mapping for the corresponding plurality of synthons for a reactant in the first plurality of reactants 86 comprises 2, 3, 4, 5, 6, 7, 8, 9, 10, 11, 12, 13, 14, 15, 16, 17, 18, 19, 20, 25, 30, 35, 40, 45, 50, or 100 or more synthons.

Referring to block 236, a molecular structure (compound) 92 is identified in the combinatorial synthesis library 82 that includes the set of synthons 88 arranged in accordance with a synthesis rule f associated with the first reaction type 82. As used herein, a synthesis rule f generates a compound x from a reaction and synthon tuple pair (t; u), e.g., x: =f (t; u).

Referring to block 238, in some embodiments, the plurality of compounds in the combinatorial synthesis library 82 comprises a billion or more compounds and the molecular structure that is identified is any one of the billion or more compounds satisfying the query. Referring to block 240, in some embodiments, the plurality of compounds in the combinatorial synthesis library 82 comprises a trillion compounds or more compounds and the molecular structure that is identified is any one of the trillion or more compounds satisfying the query. In some embodiments the combinatorial synthesis library 82 comprises 1×10⁸, 1×10⁹, 1×10¹⁰, 1×10¹¹, 1×10¹², or 1×10¹³ compounds.

### Combinatorial Synthesis Library Variational Autoencoder.

Next, consider the task of looking up a product x from a library [symbol] , which amounts to finding the reaction t and synthon tuple u satisfying x=f(t, u). This can be cast as an inference problem seeking p(t, u|x, [symbol] ). A latent variable model for x gives rise to a variational formulation:

$$p ( t , u \vert x , 𝒟 ) = {\int }_{z} p ( t , u \vert z , 𝒟 ) q ( z \vert x , dz , ) , \qquad ( 2 )$$

with p(z) denoting the prior distribution of the latent variable. One can further simplify the joint conditional distribution of t and u by first selecting the reaction t and then selecting the synthons sᵤ⁽¹⁾, . . . , sᵤ^{(k}^{ₜ}⁾ independently conditional on t:

$$p ( t , u \vert z , 𝒟 ) = p ( t \vert z , 𝒟 ) p ( u \vert z , t , 𝒟 ) \qquad ( 3 )$$

$$= p ( t \vert z , 𝒟 ) {\prod }_{i = 1}^{{k}_{t}} p ( {s}_{u}^{( i )} \vert z , t , {r}_{t}^{( i )} , 𝒟 ) . \qquad ( 4 )$$

This gives rise to a strategy in which one encodes a molecule x into a latent space z˜q (z|x) and proceeds by first decoding the reaction type t˜p(t|z, [symbol] ) and then, conditional on the sampled reaction, decoding one synthon per group sᵤ⁽ⁱ⁾˜p(sᵤ⁽ⁱ⁾|z, t, rₜ⁽ⁱ⁾, [symbol] ) for i=1, . . . , kₜ (where t is a positive integer of 2 or greater) to form the synthon tuple u∈Uₜ.

The resulting latent variable model can be called a combinatorial synthesis library variational autoencoder. FIGS. 3B-H give a step-by-step depiction of the method. Three primary modules that comprise this autoencoder are (i) a library encoder, (ii) a molecular encoder, and (iii) a molecular decoder. Module (ii) forms the basis for q (z|x), while modules (i) and (iii) form the basis for p(t, u|z, [symbol] ).

### Library Encoder.

In some embodiments, a combinatorial synthesis library [symbol] =(T, R, S, f, ψ, σ) is organized hierarchically, with synthons S at the bottom of the hierarchy, R-groups R in the middle, and reactions T at the top. A strategy is described herein for learning an associated hierarchy of representations that describe the library at these three levels of resolution in an end-to-end fashion. These representations can then be used in retrieving results from queries into the library. The library encoder is illustrated in FIGS. 3C-E.

Starting from the bottom of the hierarchy with the synthons S, to learn a representation for each synthon in a manner that is fully inductive, a graph neural network is used to parameterize a SynthonEncoder: [symbol] ₛ→ [symbol] ^{d}^{_{S}}, which applies a sequence of message passing steps followed by a readout to arrive at a d_{S}-dimensional representation for each synthon. In some embodiments the number of dimensions in d_{S} is 2, 3, 4, 5, 6, 7, 8, 9, 10, 11, 12, 13, 14, 15, 16, 17, 18, 19, 20, or more than 20 dimensions.

Moving up the hierarchy, a deep set neural network is used to represent the R-groups R by summarizing the representations for the synthons belonging to a particular group. Formally, the RGroupEncoder: [symbol] ^{d}^{_{S}}× . . . × [symbol] ^{d}^{_{S}}→ [symbol] ^{d}^{_{R}} learns a de-dimensional representation for a given R-group from the set of d_{S}-dimensional representations of the constituent synthons. Following [61], the R-group encoder has the form RGroupEncoder ({h_{S}^{S}: ∀_{S}∈σ(r)})=ρ(⊕_{seσ}(r)ϕ(h_{S}^{S})). In some embodiments, ϕ and ρ are parameterized as multi-layer perceptrons and ⊕ is a permutation-invariant aggregation operator.

In some embodiments ϕ and ρ are parameterized as a model other than a multi-layer perceptron. For instance, in some embodiments ϕ and ρ are parameterized as any of the models disclosed in the definitions section for “model” disclosed above. In some embodiments, more performant set-to-vector neural networks are used such as, e.g., set transformers [31]. However, an advantage of the use of multi-layer perceptrons is that it permits fast querying over library subsets at test time since ϕ(h_{S}^{S}) can be cached for all s∈S, thereby reducing the required computations for making queries into partitions of the library. In some embodiments, mean pooling is utilized as the aggregation operator to focus on characteristics of the distribution of synthons in a group (as opposed to sum pooling which generally expresses characteristics of the multiset), which improves performance when dealing with R-groups of varying cardinality.

In some embodiments, to represent reactions, another deep set neural network is used as a ReactionEncoder: [symbol] ^{d}^{_{R}}× . . . × [symbol] ^{d}^{_{R}}→ [symbol] ^{d}^{_{T}}. This model takes as input a variable-sized set of group representations corresponding to the reactants in t and produces a dr-dimensional representation for the reaction. In some embodiments, sum is utilized as the aggregation operator to learn multiset properties of the R-groups in a reaction.

Putting these together, the representations cascade as follows:

$${h}_{s}^{S} = SynthonEncoder ( {𝒢}_{s} ) , \qquad ( 5 )$$

$${h}_{r}^{R} = RGroupEncoder ( \{ {h}_{s}^{S} : {∀}_{S} ∈ σ ( r ) \} ) , \qquad ( 6 )$$

$${h}_{t}^{T} = ReactionEncoder ( \{ {h}_{r}^{R} : {∀}_{r} ∈ ψ ( t ) \} ) . \qquad ( 7 )$$

In some embodiments, a factorization of the likelihood is considered such that, given a molecular representation z, the molecular decoder proceeds by first decoding the reaction type and then decoding one synthon for each group separately conditional on the reaction type. Hence, in some implementations, a key vector is used for each reaction as well as for each synthon to compare with the associated query vectors. As such, a ReactionKeyGenerator is used in some embodiments that has the function [symbol] ^{d}^{_{T}}→ [symbol] ^{k}^{_{T}} to produce a key vector given a reaction representation, and similarly a SynthonKeyGenerator: [symbol] ^{d}^{_{S}}→ [symbol] ^{k}^{_{S}} to produce a key for each synthon. In some embodiments, each of these key generators is parameterized as a multi-layer perceptron:

$${k}_{t}^{T} = ReactionKeyGenerator ( {h}_{t}^{T} ) , \qquad ( 8 )$$

$${k}_{s}^{S} = SynthonKeyGenerator ( {h}_{s}^{S} ) , \qquad ( 9 )$$

In some embodiments, each of these key generators is parameterized as some other form of model. For instance, in some embodiments, each of these key generators is parameterized as any of the models disclosed in the definitions section above under the definition “models”.

### Example Training.

For a large CSL [symbol] *, encoding the entire library on each iteration of a training loop could require an excessive amount of GPU memory. To overcome this, in some embodiments a minibatch strategy is used in which a random subset [symbol] ⊂ [symbol] * is drawn from the full library according to a distribution p( [symbol] | [symbol] *). From [symbol] , synthon, R-group, and reaction representations and keys are formed. In some embodiments, a sub-sampler p ( [symbol] | [symbol] *) is used which (i) samples a subset of the reactions contained in the full library uniformly at random, keeping only the R-groups contained in the sampled reactions, and (ii) for each reaction, samples a random number of products, retaining only the synthons that are contained in the sampled products. In some embodiments teacher forcing is further used, feeding in the ground truth reaction when generating the synthon queries for the respective R-groups. Algorithm 1 below describes one embodiment of this training procedure that is used in some embodiments of the present disclosure.

Algorithm 1 - training procedure:
input Full library [symbol] *, library sub-sampler p( [symbol] | [symbol] *), batch size N, model parameters
θ, KL divergence weight β ≥ 0, choice of optimize
while topping criterion not reached do
# Prepare a minibatch
Sample a library [symbol] ~p( [symbol] | [symbol] *)
Sample a minibatch of reaction-synthon chain pairs, (tₙ, uₙ)~p(t, u| [symbol] ) for n = 1 . . . N
Form the corresponding products via synthesis, xₙ = f(tₙ, uₙ) for n = 1 . . . N
# Encode the library
Get the synthon representations, hₛ^{S} for each s ∈ S, as per equation (5)
Get the group representations, hᵣ^{R} for each r ∈ R, as per equation (6)
Get the reaction representations, hₜ^{T} for each t ∈ T, as per equation (7)
Get the reaction keys, kₜ^{T} for each t ∈ T, as per equation (8)
Get the synthon keys, kₛ^{S} for each s ∈ S, as per equation (9)
# Encode the products
Sample the product queries, zₙ for each n = 1 . . . N, as per equation (10)
Retain the KL divergence contributions, KLDₙ = D_{KL}(q(z|xₙ)||p(z)) for each n = 1 . . . N
# Decode the products with respect to the library
Get the reaction queries, [q^{T}]ₙ for n = 1 . . . N, as per equation (11)
Get the reaction probabilities, p(t|zₙ, [symbol] ) for each t ∈ T and n = 1 . . . N, as per equation (12)
$Get the synthon queries , {[ {q}_{{t}_{n} , r}^{S} ]}_{n} for each n = 1 … N , and r ∈ σ ( {t}_{n} ) , as per equation ( 13 )$
$Get the synthon probabilities , p ( {s}_{j}^{( i )} \vert {z}_{n} , {t}_{n} , {r}_{{t}_{n}}^{( i )} , 𝒟 ) for each n = 1 … N , {s}_{j}^{( i )} ∈ ψ ( {r}_{{t}_{n}}^{( i )} ) , and$
${r}_{{t}_{n}}^{( i )} ∈ σ ( {t}_{n} ) as per equation ( 14 )$
$Calculate the LL contributions , {LL}_{n} = log p ( {t}_{n} \vert {z}_{n} , 𝒟 ) + {\sum }_{i = 1}^{\vert {u}_{n} \vert} log p ( {u}_{n}^{( i )} \vert {z}_{n} , {t}_{n} , {r}_{{t}_{n}}^{( i )} , 𝒟 ) for$
each n = 1 . . . N
# Compute the loss and update parameters
$Calculate the loss as the negative of the weighted ELBO , ℓ = \frac{1}{N} {\sum }_{n = 1}^{N} β K L {D}_{n} - L {L}_{n}$
$Compute gradients {∇}_{{θ}^{ℓ}} and update θ using choice of optimizer , e . g . , θ ← optimizer ( θ , {∇}_{{θ}^{ℓ}} )$
end while
return Fitted model parameters θ* ← θ

### Ex-Post Density Estimation.

Given a trained generative model p_{θ}(x|z), products can also be sampled via (x, z)˜p_{θ}(x|z)p(z) (discarding z) (reference 3). However, due to the batch sampling strategy outlined herein, this in general does not correspond well to a uniform distribution over the products in [symbol] due to the bias introduced by the batch sampling strategy discussed above (which first uniformly samples reactions and then uniformly samples products given the reaction).

Although this can be corrected with importance weighting during the training phase, in some embodiments, another approach is performed by utilizing the following ex-post density estimation strategy [14]. A large number of products is sampled from the target distribution x˜p(x| [symbol] ) and these products are encoded via the molecular encoder z˜q_{ϕ}(z|x). A density estimator is then fitted to the aggregated samples in some embodiments, written q_{λ}(z). In some implementations, a multivariate normal distribution is utilized for simplicity, but in some embodiments a more expressive density estimator is used here (e.g., a mixture of multivariate normals).

In some implementations, products can then be sampled via (x, z)˜p_{θ}(x|z)q_{λ}(z), which will more closely align with sampling from p(x| [symbol] ). This helps to correct for bias in the distribution over product space that is induced by the choice of batch sampling strategy.

### Computational Complexity, Scalability, and Efficiency.

In this section, the computational complexity, scalability, and efficiency of some embodiments of the presently disclosed systems and methods is described.

First, it is noted that the library D can be encoded with O(|S|+|R|+|T|) complexity, where the constant depends on the complexity of the synthon, R-group, and reaction encoders. Nonetheless, this is logarithmic in comparison to naively encoding each product in [symbol] , which has O(| [symbol] |) complexity.

More noteworthy is the computational complexity of the molecular decoder. For clarity, consider a simplified D comprised of a single k-component reaction. Let Mᵢ denote the number of synthons for group i=1, . . . , k. Naively, a nearest neighbor lookup in D will have O(Πᵢ₌₁^{k}Mᵢ) complexity. In some embodiments, the presently disclosed systems and methods, on the other hand, performs the lookup with the synthons in each R-group independently, which attains O(Σᵢ₌₁^{k}Mᵢ) complexity—a logarithmic improvement. Hence, the structure of the presently disclosed molecular decoder is highly suitable for ultra-large combinatorial libraries that are of interest in early-stage drug discovery.

Another advantage of disclosed decoding strategy is that it relies only minimally on autoregression. In fact, in some embodiments only a single step of autoregression is needed irrespective of the size of the graph being generated (autoregression length of exactly two). As such, the disclosed systems and methods scale to large and variable-sized molecular graphs that follow a combinatorial synthesis construction.

Moreover, the disclosed systems and methods are guaranteed to generate chemically valid, synthetically accessible-molecular graphs without performing explicit validity checks. This compares favorably with prior work, in which the validity of each candidate action is verified at each step of the autoregression, with invalid actions excluded from the choice set. Although cheminformatics libraries like RDKit [30] have efficient C++ implementations for these checks, they nonetheless increase runtime rather significantly. Further, in the absence of explicit validity checks, these models have been shown to generate invalid molecular graphs at a markedly higher rate [23].

### Closing Remarks.

The disclosed combinatorial synthesis library variational auto-encoder is a new graph-based generative model for the navigation of combinatorial synthesis libraries. The disclosed model utilizes minimal autoregression, permitting efficient generation of large molecular graphs and improving scalability. Compounds generated by the disclosed model are chemically valid and cost-effectively accessible. In some embodiments, the disclosed model is a neural database providing random access to non-enumerable libraries. In experiments below, the capabilities of the disclosed model in modeling ultra-large and realistic make-on-demand libraries is demonstrated, paving a path towards more scalable strategies in the exploration of non-enumerable chemical libraries for early-stage drug discovery.

In some embodiments, the disclosed synthon lookup in the decoder scales linearly with the number of synthons in an R-group, which can present challenges as libraries continue to add many synthons per R-group. In some embodiments this is mitigated with more scalable query-key designs [11, 26]. Furthermore, in some embodiments because of the presence of prominent R-group symmetry (e.g., as in polymers) some embodiments of the decoder include modifications to break parity, and some such modification may not admit the same convenient parallelization. Lastly, softmax has limitations in mapping from real-valued potentials to chosen probabilities due to its rigid substitution patterns [55]. Accordingly, in some embodiments, rather than using softmax, sparse or alternative-aware softmax variants are used.

Synthon encoder. In some embodiments, the synthon encoder (SynthonEncoder) is a message passing graph neural network, taking node, edge, and graph features as input, and producing updates to these features after every round of message passing in a residual fashion. A single message passing layer below.

Preliminaries. A graph [symbol] =(V, ε) is represented by a set of node features {xᵢ⁰: i∈V}, edge features {eᵢⱼ⁰: (i,j)∈ε}, and graph features g⁰. Superscripting by zero indicates that these are the initial or input features; below, superscripting by [symbol] denotes the result after the [symbol] th round of message passing.

Edge model. The edge model updates the edge features as follows. For an edge (i, j) ∈ε, the current iterate [symbol] of the edge features, graph features, and the two corresponding node features are concatenated. The concatenated features are then layer normalized and processed by a two-layer MLP, producing

$$Δ {e}_{i j}^{ℓ + 1} = Linear ( ReLU ( Linear ( ReLU ( LayerNorm ( [ {e}_{i j}^{ℓ} ‖ {g}^{ℓ} ‖ {x}_{i}^{ℓ} ‖ {x}_{i}^{ℓ} ] ) ) ) ) ) .$$

The output dimension of the final linear layer is set to be equal to the dimensionality of the edge features, which allows the edge features to be updated in a residual fashion, e.g.,

$${e}_{i j}^{ℓ + 1} = {e}_{i j}^{ℓ} + Δ {e}_{i j}^{ℓ + 1} .$$

Note that for an edge (i, j)∈ε, edge features are separately maintained for i→j and i←j updates; these features are identical for [symbol] =0, but are not the same in general for subsequent layers.

Node model. Given a node i∈V, a message is formed from all its neighboring nodes by summing over the associated incoming edge features,

$${m}_{i}^{ℓ + 1} = {\sum }_{j ∈ 𝒩 ( i )} {e}_{i j}^{ℓ + 1} .$$

Similar to the edge model, the node feature, message, and graph feature are concatenated, layer normalized, and passed through a two-layer MLP in which the final linear layer has output dimension equal to the dimensionality of the node features. This produces a residual update for the node feature, given by

$$Δ {x}_{i}^{ℓ + 1} = Linear ( ReLU ( Linear ( ReLU ( LayerNorm ( [ {x}_{i}^{ℓ} ‖ {m}_{i}^{ℓ + 1} ‖ {g}^{ℓ} ] ) ) ) ) ) .$$

The node features are thus updated according to

$${x}_{i}^{ℓ + 1} = {x}_{i}^{ℓ} + Δ {x}_{i}^{ℓ + 1} .$$

Graph model. The graph features are updated by accumulating messages from each node in V as follows. Node messages to the graph

$$\{ {n}_{i}^{ℓ + 1} : i ∈ V$$

are formed using the now familiar layer norm+MLP design, with the final linear layer having output dimension equal to the dimensionality of the graph feature. These messages,

$${n}_{i}^{ℓ + 1} = Linear ( ReLU ( Linear ( ReLU ( LayerNorm ( [ {x}_{i}^{ℓ} ‖ {m}_{i}^{ℓ + 1} ‖ {g}^{ℓ} ] ) ) ) ) ) ,$$

are then aggregated via sum pooling to form a residual to the graph features,

$$Δ {g}^{ℓ + 1} == {\sum }_{i ∈ 𝒱} {n}_{i}^{ℓ + 1} ,$$

which updates the graph features accordingly,

$${g}^{ℓ + 1} = {g}^{ℓ} + Δ {g}^{ℓ + 1} .$$

Implementation-wise, the node and graph model use a shared MLP with the output dimension equal to the sum of the node and graph feature dimensions, and the outputs are then split into two parts: one which routes to the node update, and the other to the graph update.

Putting it all together. The message passing neural network applies a sequence of message passing layers as outlined above. In one implementation in accordance with the present disclosure, the node, edge, and graph feature dimensions are set to 64 and four message passing layers are utilized. For the input graph features, in some embodiments a vector of zeros is used. The final graph features serve as the synthon representations, which are used in producing synthon keys and the cascaded representations for the R-groups and reactions. In total, one embodiment of the SynthonEncoder in accordance with the present disclosure is described by 152,832 parameters.

R-group encoder. In some embodiments in accordance with the present disclosure the R-group encoder (RGroupEncoder) follows the design outlined in DeepSets, in which the synthon representations are each processed separately by an MLP, pooled together (here, a mean pooling is used to allow the network to focus on characteristics of the distribution of synthons belonging to an R-group), and the result processed by yet another MLP. In some embodiments in accordance with the present disclosure, both MLPs are set as two-layer networks with ReLU activation in between the two linear operations. In some embodiments, all dimensions are 64, and as such the RGroupEncoder utilizes 16,640 parameters in total. In some alternative embodiments, any of the models disclosed in the definitions section above are used to construct the R-group encoder.

Reaction encoder. In some embodiments, the reaction encoder (ReactionEncoder) follows the same design as the RGroupEncoder, with the exception that sum pooling is utilized instead of mean pooling to allow the network to focus on the multi-set of R-groups in a reaction. In some embodiments, all dimensions are 64, and as such the ReactionEncoder also utilizes 16,640 parameters in total. In some alternative embodiments, any of the models disclosed in the definitions section above are used to construct the reaction encoder.

Synthon key generator. In some embodiments, the synthon key encoder (SynthonKeyGenerator) is an MLP that produces a synthon key from a synthon representation. In some embodiments, a linear layer is used. In some embodiments, the input and output dimensions are both set to 64, and so, in such embodiments the SynthonKeyGenerator utilizes 4,160 parameters in total. In some alternative embodiments, any of the models disclosed in the definitions section above are used to construct the synthon key encoder.

Reaction key generator. In some embodiments, the reaction key generator (ReactionKeyGenerator) is an MLP that produces a reaction key from a reaction representation. In some embodiments, a linear layer is used. In some embodiments, the input and output dimensions are both set to 64, and in some embodiments the ReactionKeyGenerator utilizes 4,160 parameters in total. In some alternative embodiments, any of the models disclosed in the definitions section above are used to construct the reaction key generator.

Molecular encoder. In some embodiments, the molecular encoder (MolecularEncoder) produces molecular queries and utilizes the same message passing design as the SynthonEncoder. In some such embodiments the node, edge, and graph features are all set to 64, and four layers of message passing are used. However, in some embodiments, differently from the SynthonEncoder, the MolecularEncoder has an additional variational linear layer that produces a conditional mean and conditional log variance vector from the graph features produced by the final message passing round. In some embodiments, the variational linear layer takes a 64 dimensional graph feature as input and produces a 128 dimensional output, which is split into the mean and log variance portions. In some embodiments, the MolecularEncoder utilizes a total of 152,832+8,320=161,152 parameters. In some alternative embodiments, any of the models disclosed in the definitions section above are used to construct the molecular encoder.

Molecular query processing network. In some embodiments, the molecular queries are regularized to be close to the prior p (z) as a consequence of the VAE objective. To allow the decoder to make better use of these features, in some embodiments the molecular queries are processed by an MLP prior to being passed to the reaction and synthon query generators. Opting for simplicity, in some embodiments a simple two-layer MLP with intermediate ReLU activation; all dimensions are 64, which results in 8,320 parameters, is used. In some embodiments, any of the models disclosed in the definitions section above are used instead of such an MLP.

Reaction query generator. In some embodiment, the reaction query generator (ReactionQueryGenerator) is an MLP that produces a reaction query from the molecular query. In some embodiments, a two-layer network with intermediate ReLU activation is used. In some embodiments, all dimensions are set to 64, so the ReactionQueryGenerator utilizes 8,320 parameters in total. In some embodiments, any of the models disclosed in the definitions section above are used instead of such an MLP for the reaction query generator.

Synthon query generator. In some embodiments, the synthon query generator (SynthonQueryGenerator) is an MLP that produces a synthon query from the molecular query, reaction representation, and R-group representation. In the general case, in some embodiments these three feature types are concatenated when a common dimensionality of 64 has been used throughout, and to keep implementation simple, and a sum is used instead of concatenation in some embodiments (which can be shown to be equivalent to concatenating with an additional constraint on the weight matrix for the subsequent linear layer). Like the ReactionQueryGenerator, in some embodiments a two-layer MLP with intermediate ReLU activation, resulting in a total of 8,320 parameters for the SynthonQueryGenerator. In some alternative embodiments, any of the models disclosed in the definitions section above are used instead of such a MLP for the synthon query generator.

### EXAMPLES

### Example 1—Comparison of Disclosed Model to JT-VAE and RationaleRL

### Data.

To demonstrate the capabilities of the presently disclosed systems and methods on the kind of real-world combinatorial synthesis catalogs that are employed in large-scale hit discovery programs today, example experiments were performed that utilized the Enamine REadily AccessibLe (REAL) library, which is comprised of 340K synthons and over a thousand reactions. The reactions in REAL range from two to four components and the number of synthons per R-group can range from the single digits to tens of thousands. In total, the REAL library describes a chemical space of over 16 billion commercially available compounds [4] which can be acquired at low cost on the order of three to four weeks. Advantageously, the use of this library alongside the presently disclosed systems and methods can be used to reproduce the example experiments and to foster further research in the machine learning community on combinatorial synthesis libraries.

### Training.

During training, subsets of the library were sampled as follows. Of the roughly 1300 reaction types in the REAL database, 20 reactions were first uniformly sampled at random, and subsequently 100 products per reaction were sampled, including the associated synthons in the library subset. These library subsets therefore describe roughly 300K-1.5M compounds each, which is significantly smaller than the complete library of 16 billion compounds. See Algorithm 1 above for details.

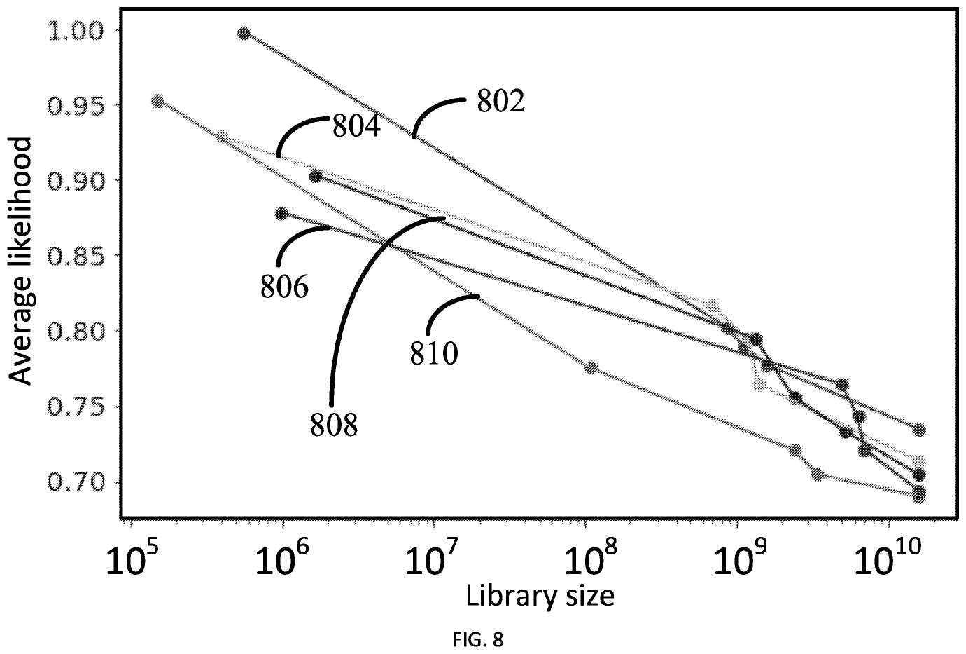

Testing. For test-time inference, decoding is with respect to the full library of 16 billion compounds. This constitutes a test-time distribution shift relative to training, but it was observed that the disclosed systems and methods generalizes well to the full library without modifications. For completeness, an analysis of the test-time distribution shift is provided in FIG. 8.

As described in Algorithm 1, small subsets of the full library are sampled in each training iteration for tractability. In particular, the library sub-sampler utilized first samples uniformly over the reactions and subsequently samples a constant number of products in each reaction at random; the synthons associated with these sampled products comprise the synthons in the library subset. In training the CSL VAE model used in this experiment, 20 reactions and 100 products per reaction are sampled, which yields a minibatch of 2000 products. As described herein, these associated library subsets describe a chemical space of roughly 300K-1.5M compounds each. At test time, however, decode according to the full library of 16B compounds is done, which constitutes a fairly drastic test-time distribution shift. FIG. 8 illustrates the magnitude of the test-time distribution shift on reconstruction quality (measured by average likelihood). Starting by sampling five library subsets as described above, drawing 2000 compounds for each subset; each of these five draws is represented by a different line in the figure. These library subsets are then gown by first including all synthons across the 20 sampled reactions and calculating the average reconstruction likelihood for the same 2000 products according to the expanded library, and further increasing the library by adding all synthons for 100 randomly sampled additional reactions, and then all synthons for yet another 100 randomly sampled additional reactions, and finally including all synthons and all reactions. At each step, the library subsets describe an increasingly larger chemical space. It is observed that the average likelihood decreases in a roughly linear-log fashion in the library size as a result of this distribution shift.

### Molecular Reconstruction and Generation.

An embodiments of the disclosed system and method (CSLVAE) was compared against two existing molecular graph generative models: JT-VAE and RationaleRL [25]. All three models were trained from scratch on the Enamine REAL library.

In JT-VAE, molecular graphs are represented by junction trees over chemical fragments. Decoding proceeds by first generating the junction tree in a depth-first manner, placing a fragment in each node, and then subsequently orienting the fragments to match attachment points. RationaleRL, on the other hand, takes as input a starting rationale. The decoder's objective is to complete the molecule in an autoregressive fashion (one graph edit per step). In the present example, a product from the library is taken and all but one synthon removed, treating the resulting graph as the starting rationale. Thus, RationaleRL is tasked with generating the missing synthon in the present example.

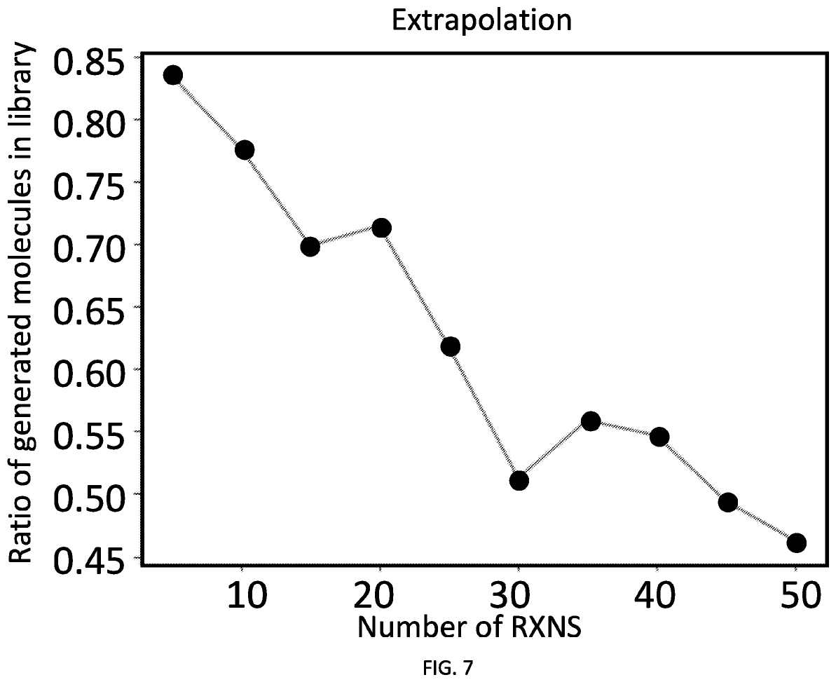

Table 3 summarizes the key findings from this exercise. First, it is noted that the implementation of the disclosed systems and methods utilized has roughly 10× fewer parameters than the two alternatives considered, owing to the inductive nature of the library encoder. All three methods achieve 100% chemical validity, but the disclosed systems and methods achieves this result without explicit validity checks. The average likelihood is computed by taking the average of per-compound reconstruction likelihoods across a large number of products sampled from the library. This is a measure of how well the model is capable of reconstructing the full molecular graph (e.g., on average, how likely it is to reproduce the query molecule via the decoder) and can also loosely be interpreted as a measure of coverage/reachability (e.g., what percent of the library is it able to faithfully cover). Finally, the example experiments highlight the challenges existing graph generative models face when applied to ultra-large combinatorial synthesis libraries, namely that they struggle to reliably generate in-library compounds. For JT-VAE, fewer than 1 in 34 compounds were found in REAL. RationaleRL, on the other hand, generates in-library completions in only about half of the cases (see FIG. 7), but has the advantage that it is provided a starting rationale in the form of a compound from REAL stripped of all but one synthon. In contrast, the disclosed systems and methods guarantee that the result is from the library by design.

TABLE 3
Comparison of RationaleRL, JT-VAE, and CSLVAE
on synthon-based generative modeling

| | JT-VAE | RationaleRL | CSLVAE |
|---|---|---|---|
| # Parameters | 4.7M | 3.4M | 380K |
| Validity | 100.0% | 100.0% | 100.0% |
| Uniqueness | 80.1% | 96.3% | 98.8% |
| Average likelihood | 18.7% | 62.3% | 72.4% |
| In-library proportion | 2.9% | 50.9% | 100.0% |

### Dataset Preparation.

The Enamine REAL library is a combinatorial synthesis library of roughly 1300 reaction types, ranging from 2- to 4-component reactions, along with roughly 340K synthons. In total, the REAL library describes a chemical space in excess of 16B make-on-demand compounds. For the two baseline models of the present comparisons, memory issues arose when using the author-provided code on the full REAL library (both in the vocabulary generation steps as well as with writing products to disk). As such, the two baselines were trained and evaluated on a subset of the full REAL library. Memory limitations were not faced with CSLVAE (the model of the present disclosure), which was trained on the full library and evaluated as such. Hence, RationaleRL and JT-VAE can be compared on a per-item basis (each having been trained on the same subset of REAL), whereas the results for CSLVAE reflect training on the larger and more diverse full REAL library. RationaleRL and JT-VAE were not developed with the goal of searching through combinatorial synthesis libraries, and the use of them in this comparison serves as an attempt to compare the disclosed method (CSLVAE) with the application of existing state-of-the-art graph generative model on combinatorial synthesis libraries out-of-the-box and without modification.

To construct the data on which RationaleRL and JT-VAE were trained, the 1300 reaction types were ranked by the number of products contained in each reaction and selected 50 of the middle-sized reactions. In total, these reactions describe a chemical space of 125M compounds. The products were sampled from each reaction such that all synthons are represented, amounting to a training set of ˜500K compounds.

### RationaleRL Details.

The pre-training phase of RationaleRL was utilized, which trains a graph-based variational autoencoder that seeks to reconstruct a molecular graph from a starting rationale (and does not require RL, as the name might suggest; that is part of the fine-tuning phase, which is not utilized here). Given the starting rationale and full molecule, RationaleRL's decoder completes the molecule autoregressively in an atom-by-atom, bond-by-bond fashion. To form the starting rationale, the product from REAL is taken and all but one synthon is removed. Hence, RationaleRL is tasked with completing the missing synthon given the full molecule (as input to the encoder) and the starting rationale (as input to the decoder, along with the latent code). In FIG. 7, it is shown that as the number of distinct reactions in training is increased, the probability that completions generated by RationaleRL yield a product from the library drops precipitously; after 50 reactions are included, fewer than half of all completions yield a product in the REAL library. These experiments are based on the author-provided code, which can be found at https://github.com/wengong-jin/multiobj-rationale.

### JT-VAE Details.

JT-VAE is a graph-based variational autoencoder that generates molecules according to a tree-structured scaffold of fragments. Unlike RationaleRL, JT-VAE samples chemical fragments in an autoregressive fashion, rather than atoms or bonds. Prior to training, the vocabulary generation step described in the JT-VAE paper was applied to the sampled products from the subset of REAL utilized in the baseline experiments, which yields a total of 325 fragments. This experiments is based on the author-provided code, which can be found at https://github.com/wengong-jin/icml18-jtnn.

### Details of the Disclosed Model (CSLVAE).

During training we an annealing schedule on β was utilized (see Algorithm 1), starting with β=0 and incrementing by 1e-5 every 2000 iterations, with a max value of β=1. Training was incurred for a total of 200K iterations, in which time CSL VAE has seen a total of 2000 200K=400M compounds (although not 400M unique compounds, due to the batch sampling strategy). As such, by the time training is halted, CSLVAE has seen no more than 2.5% of the full REAL library.

### Example 2—Latent Space Visualizations

The latent space learned by the discloses systems and methods was qualitatively inspected. Of interest was verifying whether the proposed model has learned a latent space that varies relatively smoothly over the covered chemical space (e.g., that small perturbations to the query induce only minor edits in the resulting molecular graph). Two kinds of checks were performed: latent space interpolations and local neighborhood visualizations.

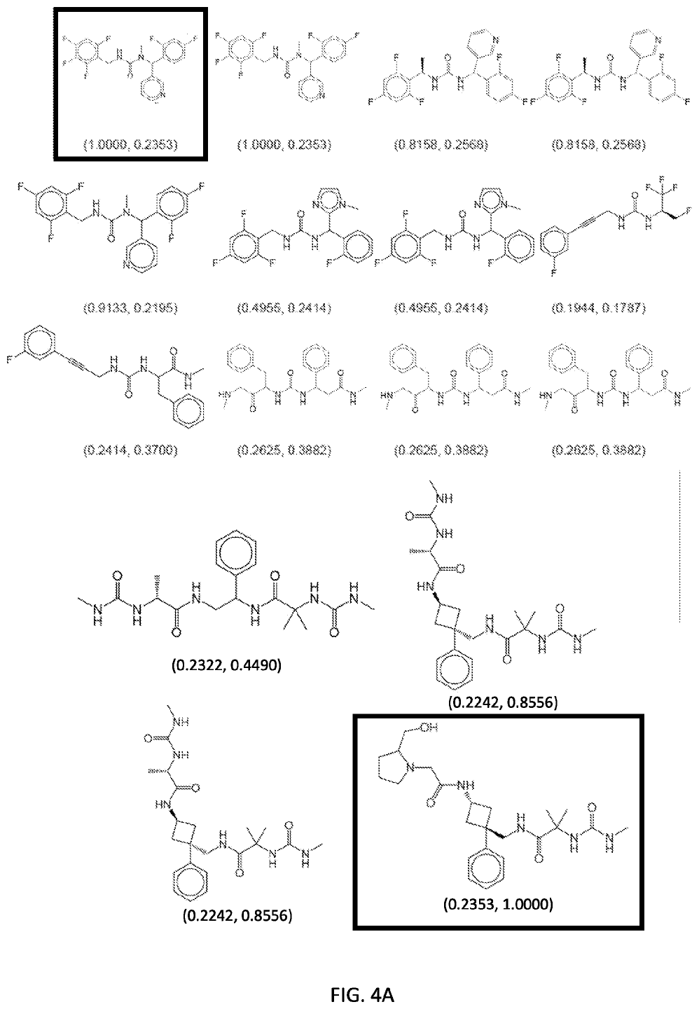

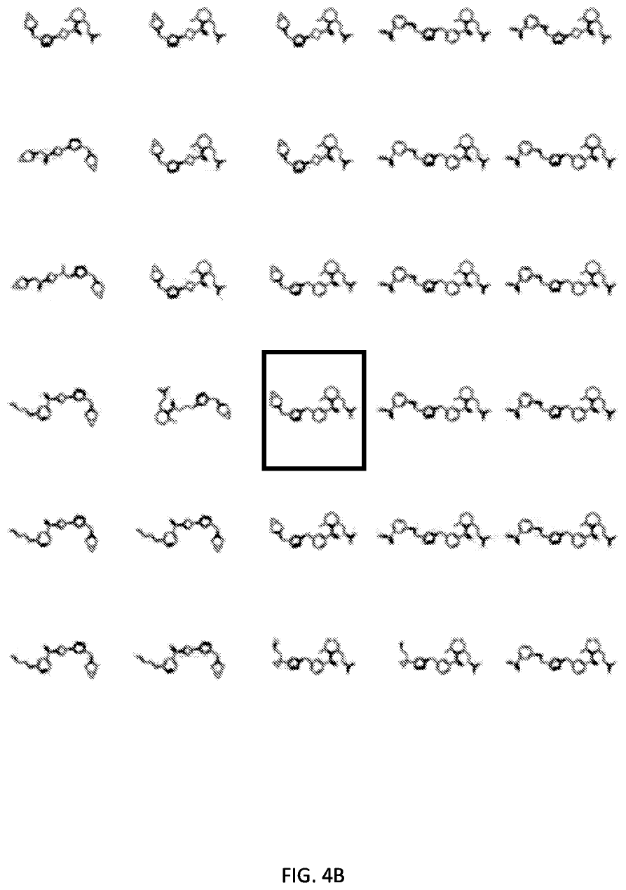

FIG. 4A contains an example of interpolations in the latent space. In particular, the molecular queries for the starting compound (top left) and target compound (bottom right) were interpolated, in raster order. The molecules immediately adjacent to the starting and target compounds are the associated reconstructions. Products are decoded with respect to the full REAL library of 16 billion compounds. Below each molecule in FIG. 4A are its Tanimoto similarity (5) with the starting and target molecule, respectively. It is observed that the interpolations traverse through regions of chemical space that gradually decrease (cf. increase) in similarity with respect to the starting (cf. target) compound.

FIG. 4B visualizes the latent space around a randomly sampled product from the REAL library. Following prior work [29], a random 2D plane was formed in the high-dimensional latent space by sampling two random directions around the molecular query (center compound) and decoding the resulting products using the argmax decision rule. It is observed that the latent space is smooth in the sense that molecules morph gradually, with only minor edits when the movement in latent space is small (e.g., one synthon at a time, modifications to smaller functional groups), and that the molecular scaffold is generally conserved locally.

### Example 3—Analog Retrieval Via Autoencoding

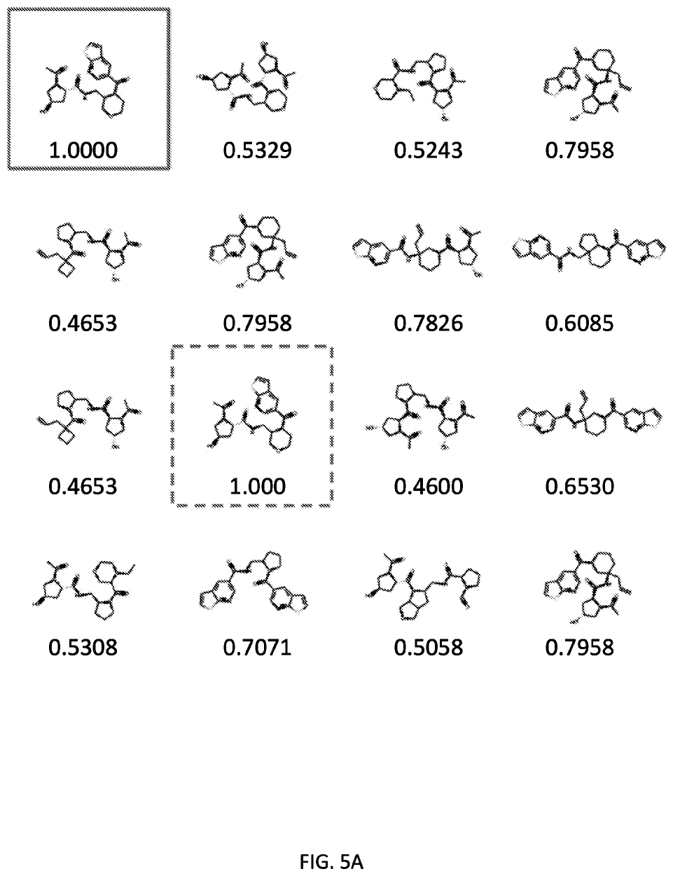

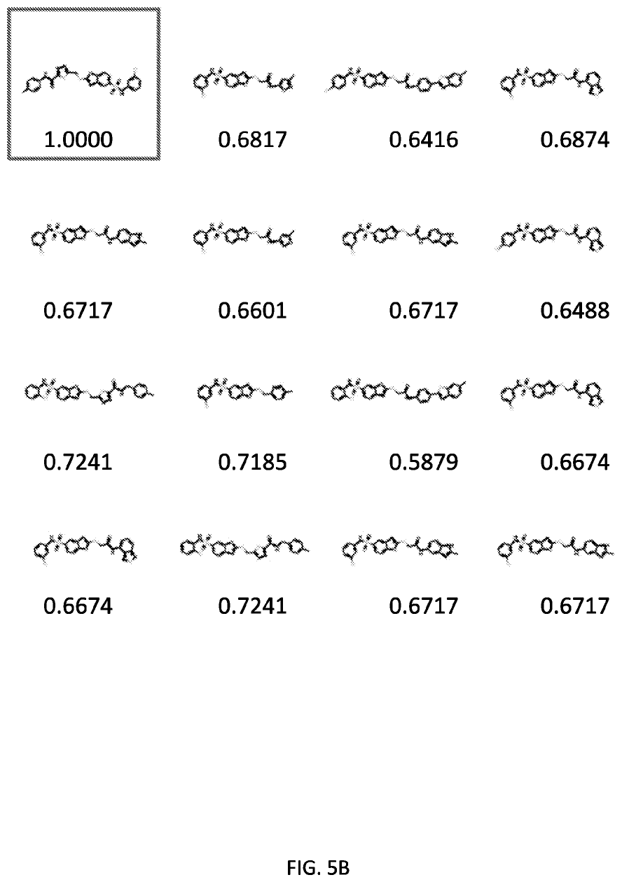

The discloses systems and methods was used to find analogs of a query compound in a large CSL. In FIGS. 5A and 5B the disclosed models of the present disclosure are presented with two classes of molecules: ones that are in the library (FIG. 5A) and ones that are not in the library (FIG. 5B). Each query molecule (top left) of FIG. 5A and (top left) of FIG. 5B is encoded and stochastically decoded fifteen times using the disclosed model (e.g., illustrated in FIGS. 1A, 1B, and 3). Thus, given the molecular query, conditionally random completions are generated from the decoder. In both cases, autoencoding retrieved highly similar compounds as measured by Tanimoto similarity. For the in-library example, the model is able to successfully retrieve the query compound (third row, second column of FIG. 5A). Moreover, it is observed that many instances in which autoencoding returns compounds that have the same reaction type as the query molecule but vary in one or two synthons. For the out-of-library example, the disclosed systems and methods find skeletally relevant compounds with high Tanimoto similarity, indicating the utility of this approach for fast analog search in large (non-enumerable) libraries.

### Example 4—Ex-Post Density Estimation

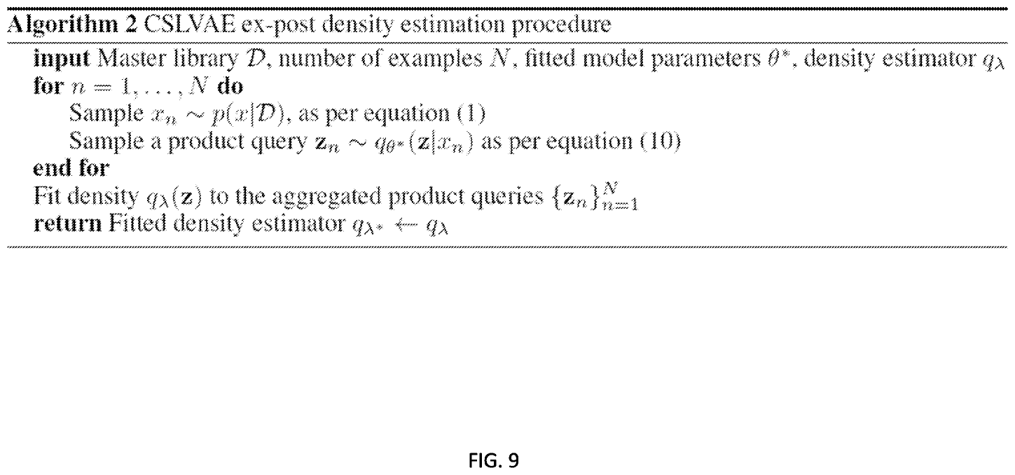

The use of ex-post density estimation on latent codes corresponding to products sampled uniformly from the library as a way to force random samples from CSLVAE is disclosed above to track more closely to sampling uniformly at random from the library. Algorithm 2, found in FIG. 9, outlines this procedure.

An experiment was carried out demonstrate that Algorithm 2 achieves the intended result. The experiment proceeds as follows. 10,000 molecules are drawn from the library uniformly at random and are treated as a training set for the ex-post density estimator. Three density estimators of increasing expressivity are considered: a multivariate normal (MVNormal), a mixture of five normals (MoG-5), and a mixture of ten normals (MoG-10). The standard approach of sampling from the isotropic multivariate normal prior compared. As such, four alternative schemes for sampling latent codes that are subsequently decoded into compounds from the library are used in this experiment. Another 10,000 molecules are then sampled from the library, again uniformly at random, which is treated as a reference set for comparison.

TABLE 4
Divergences between query sampling strategies and a reference
set of compounds sampled uniformly at random from the library.

| | Train | Prior | MVNormal | MoG-5 | MoG-10 |
|---|---|---|---|---|---|
| Uniqueness | 100% | 96.9% | 99.8% | 99.7% | 99.8% |
| SA (JSD) | 0.0179 | 0.3340 | 0.0651 | 0.0557 | 0.0452 |
| QED (JSD) | 0.0134 | 0.2200 | 0.0572 | 0.0266 | 0.0225 |
| MW (JSD) | 0.0169 | 0.4370 | 0.0970 | 0.0650 | 0.0686 |
| logP (JSD) | 0.0144 | 0.0259 | 0.0643 | 0.0686 | 0.0703 |

Following [41], the aforementioned sets of generated molecules are compared to the reference set of molecules on the following computable molecular properties: synthetic accessibility (SA), quantitative estimation of drug-likeness (QED), molecular weight (MW), and logarithm of the octanol-water partition coefficient (logP). In particular, the Jensen-Shannon distance is calculated (square root of the Jensen-Shannon divergence) between the distribution of these properties on the reference set and each set in question. Table 4 summarizes the results of this exercise. Three items worth noting are that (a) the reference compounds and the compounds sampled for training the ex-post density estimators have low divergence across the various properties, (b) sampling from the prior generates compounds with high divergence relative to uniform sampling over the library, and (c) using more expressive density estimators for the latent codes leads to increasingly lower divergence across the various properties with the reference set, as they are able to better match the distribution of latent codes for the training set (which is exchangeable with the reference set).

### Example 5—Comparison to Existing Analog Enumeration Approaches

An attempt to compare CSLVAE's analoging capabilities with that of Arthor, a state-of-the-art commercial similarity search tool for synthesis libraries developed by NextMove Software was performed in Example 5. Arthor performs analog enumeration using a custom ECFP4 bit vector representation of molecules, returning compounds with high Tanimoto similarity according to this fingerprint. For a given query compound, the top-100 analogs returned by Arthor from REAL were enumerated. Similarly, each query compound was encoded with the CSL VAE encoder and a corresponding 100 stochastic decodings was generated. For every analogue, its RDKit ECFP4 Tanimoto similarity was computed with the query compound and the top-1 analog was retained. The distribution of Tanimoto similarities of the top-1 analogs returned in this way was compared between Arthor and CSLVAE. As a control, a naive random baseline policy was used that samples 100 compounds at random from REAL, again selecting the top-1 analogue based on RDKit ECFP4 Tanimoto similarity. Because both CSLVAE and the random baseline constitute stochastic policies, this procedure was repeated 30 times for each query compound and the average top-1 Tanimoto similarity was taken.

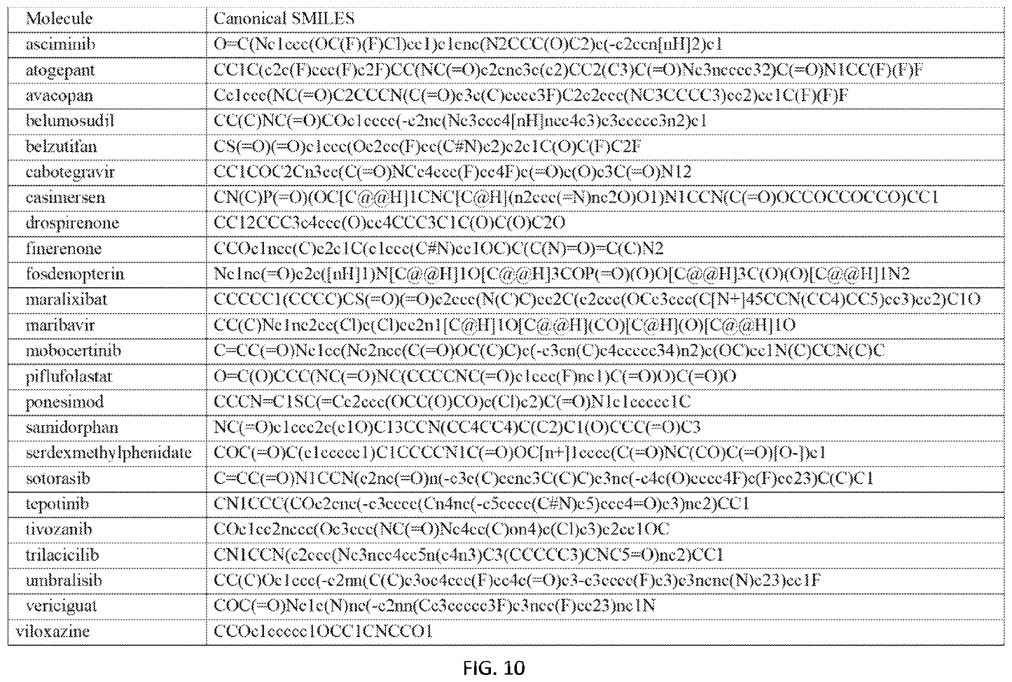

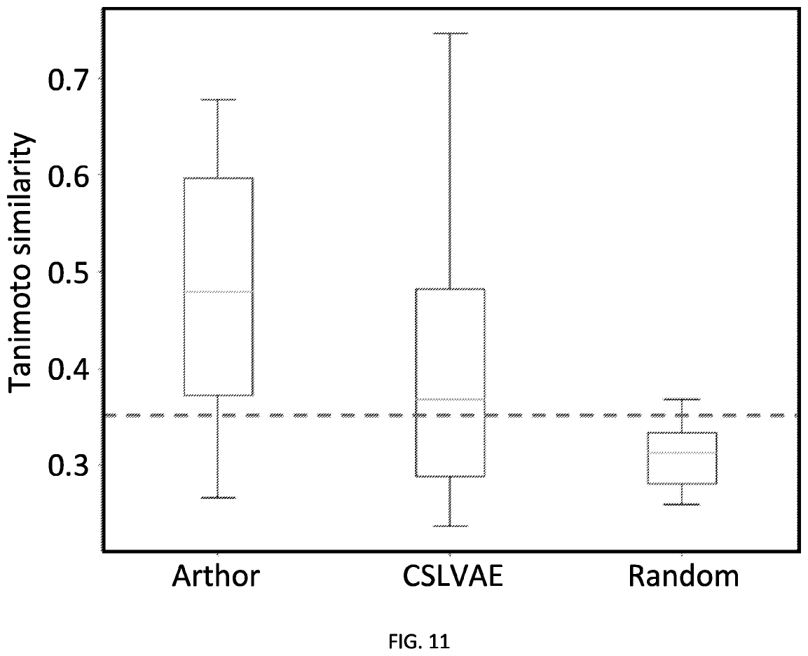

For the query compounds, 24 of the 51 novel drugs approved in 2021 by the FDA were used, filtering out drugs that do not satisfy a routine set of small molecule criteria (such as monoclonal antibodies); the compounds used in this experiment are shown in FIG. 10. FIG. 11 shows a boxplot of the top-1 Tanimoto similarities for Arthor, CSLVAE, and the random baseline.

While CSLVAE finds more distant ECFP4 analogues compared to Arthor (which is a gold standard for fingerprint similarity search), it is nonetheless able to identify analogs for unseen, novel drugs in a routine manner. In this regard, it is typical to use a Tanimoto similarity threshold in the range 0.3-0.35 to indicate whether a pair of molecules can be seen as analogues

Of particular note, CSLVAE is considerably less resource intensive than Arthor, which is specially designed for fast and efficient fingerprint-based analogue enumeration and requires appropriate infrastructure and setup. For these reasons, it is difficult to do a direct apples-to-apples comparison of the compute requirements. Nevertheless, the following pertinent information was derived from this experiment. The CSLVAE experiments were run on a machine with an NVIDIA Tesla K80 GPU and an Intel Xeon E5-2686 CPU. On average, sampling 100 analogs for a given query using CSLVAE completed in 11.41 seconds with this setup (encoding and decoding). Further, all of the parameters and buffers (including representations and keys for the synthon, R-group, and reactions) of the trained CSLVAE model required just 170 MB memory. By comparison, the Arthor experiments are run in a distributed fashion on 100 pods, each with 8 CPUs, with top-100 analogue enumeration requiring a total of 32.46 seconds, for an approximate total CPU time of 25,968 seconds. Furthermore, Arthor needs to devote rather significant amounts of memory and storage to carry out analog enumeration; our in-house setup uses 3 GB per shard and makes 64 GB RAM requests for each Arthor worker.

We note that CSLVAE's ability to represent large CSLs with significantly fewer resources and perform analog retrieval with notably improved execution time (perhaps three orders of magnitude faster) owes to its decoding strategy, which utilizes parallel synthon look-ups, thereby requiring a number of keys that is on the order of number of synthons in the library rather than on the order of number of products in the library, and further permits a kind of similarity search in time that is logarithmic in the number of products in the library (rather than linear).

### Example 6—Encoder Transfer to Molecular Property Prediction

The CSLVAE training objective can be viewed as a kind of contrastive pretext task, which seeks to align the representation of a given molecule with representations that correspond to retrieval instructions in a CSL. Hence, it is natural to wonder whether the encoder of a trained CSLVAE model could demonstrate good transfer performance in prediction tasks that may be of interest. To investigate, MLPs trained on CSLVAE were compared with those trained on molecular fingerprints (ECFP4 and ECFP6) for molecular property prediction tasks. The octanol-water partition coefficient (logP) and the quantitative estimate of drug likeness (QED) [6] were used as targets for prediction.

For this experiment, a dataset was constructed by sampling 100K compounds uniformly at random from REAL, splitting the examples into training, validation, and testing folds using an 80-10-10 split. For each compound, its CSLVAE query was extracted as its feature descriptor, in addition to its ECFP4 and ECFP6 fingerprints. Using the training fold, an MLP was fit on each of these feature descriptors separately to predict the molecule's logP and QED score. The iteration that attains the lowest validation RMSE was selected, recording its test RMSE. This was repeated five times, and the average test RMSE and standard deviation was reported. To demonstrate the extent to which CSLVAE learns molecular features that are predictive of such molecular properties for out-of-domain compounds, this exercise was repeated on a dataset of 250K molecules from ZINC. The results of this exercise are summarized in Table 5.

TABLE 5
Encoder transfer on logP and QED prediction. The cells report the
average RMSE ± one standard deviation, calculated over five runs.

| | | REAL | ZINC | REAL | ZINC |
|---|---|---|---|---|---|
| | | 100K | 250K | 100K | 250K |
| | Dimensionality | logP | logP | QED | QED |
| CSLVAE | 64 | 0.539 ± 0.002 | 0.591 ± 0.001 | 0.072 ± 0.001 | 0.068 ± 0.001 |
| ECFP4 | 256 | 0.827 ± 0.001 | 0.679 ± 0.002 | 0.091 ± 0.002 | 0.079 ± 0.001 |
| ECFP6 | 1024 | 0.601 ± 0.002 | 0.490 ± 0.001 | 0.072 ± 0.001 | 0.064 ± 0.001 |

The results of this experiment confirm that the latent space learned by CSLVAE can indeed be utilized to success in predicting quantities like logP and QED, especially in the in-domain case where it out-performs the predictors fit on chemical fingerprints. However, in the out-of-domain case, the predictor fit on ECFP6 fingerprints performs notably better than the predictor fit on CSLVAE queries, suggesting that the features learned by CSLVAE may be missing some pertinent predictive information about input molecules which differ significantly from the CSL on which it was trained.

### Example 7—Summary of CSLVAE Architecture Used in the Examples

Table 6 summarize the number of parameters used in the example CSLVAE architecture for the examples of the present disclosure.

TABLE 6
Summary of CSLVAE architecture used in the examples

| | | Number of |
|---|---|---|
| Module | Module type | parameters |
| Atom embedding | Embedding | 3,200 |
| Bond embedding | Embedding | 768 |
| Synthon encoder | GNN | 152,832 |
| R-group encoder | DeepSets | 16,640 |
| Reaction encoder | Deep Sets | 16,640 |
| Synthon key generator | Linear | 4,160 |
| Reaction key generator | Linear | 4,160 |
| Molecular encoder | GNN | 161,152 |
| Molecular query | MLP | 8,320 |
| processing network | | |
| Reaction query generator | MLP | 8,320 |
| Synthon query generator | MLP | 8,320 |
| | | 384,512 |

### REFERENCES

[1] Atanu Acharya, Rupesh Agarwal, Matthew B Baker, Jerome Baudry, Debsindhu Bhowmik, Swen Boehm, Kendall G Byler, SY Chen, Leighton Coates, Connor J Cooper, et al. Supercomputer-based ensemble docking drug discovery pipeline with application to COVID-19. Journal of Chemical Information and Modeling, 60 (12): 5832-5852, 2020.
[2] Josep Arus-Pous, Thomas Blaschke, Silas Ulander, Jean-Louis Reymond, Hongming Chen, and Ola Engkvist. Exploring the GDB-13 chemical space using deep generative models. Journal of Cheminformatics, 11 (1): 1-14, 2019.
[3] Dávid Bajusz, Anita Rácz, and Károly Héberger. Why is Tanimoto index an appropriate choice for fingerprint-based similarity calculations? Journal of Cheminformatics, 7 (1): 1-13, 2015.
[4] Louis Bellmann, Patrick Penner, and Matthias Rarey. Topological similarity search in large combinatorial fragment spaces. Journal of Chemical Information and Modeling, 61 (1): 238-251, 2020.
[5] Andreas Bender and Robert C Glen. Molecular similarity: a key technique in molecular informatics. Organic and Biomolecular Chemistry, 2 (22): 3204-3218, 2004.
[6] G Richard Bickerton, Gaia V Paolini, Jérémy Besnard, Sorel Muresan, and Andrew L Hopkins. Quantifying the chemical beauty of drugs. Nature Chemistry, 4 (2): 90-98, 2012.
[7] Lorenz C Blum and Jean-Louis Reymond. 970 million druglike small molecules for virtual screening in the chemical universe database GDB-13. Journal of the American Chemical Society, 131 (25): 8732-8733, 2009.
[8] John Bradshaw, Brooks Paige, Matt J Kusner, Marwin Segler, and José Miguel Hernández-Lobato. A model to search for synthesizable molecules. Advances in Neural Information Processing Systems, 32, 2019.
[9] John Bradshaw, Brooks Paige, Matt J Kusner, Marwin Segler, and José Miguel Hernández-Lobato. Barking up the right tree: an approach to search over molecule synthesis DAGs. Advances in Neural Information Processing Systems, 33:6852-6866, 2020.
[10] Hongming Chen. Can generative-model-based drug design become a new normal in drug discovery? Journal of Medicinal Chemistry, 65 (1): 100-102, 2021.
[11] Krzysztof Choromanski, Valerii Likhosherstov, David Dohan, Xingyou Song, Andreea Gane, Tamas Sarlos, Peter Hawkins, Jared Davis, Afroz Mohiuddin, Lukasz Kaiser, et al. Rethinking attention with performers. arXiv preprint arXiv: 2009.14794, 2020.
[12] Hanjun Dai, Yingtao Tian, Bo Dai, Steven Skiena, and Le Song. Syntax-directed variational autoencoder for structured data. arXiv preprint arXiv: 1802.08786, 2018.
[13] Wenhao Gao and Connor W Coley. The synthesizability of molecules proposed by generative models. Journal of Chemical Information and Modeling, 60 (12): 5714-5723, 2020.
[14] Partha Ghosh, Mehdi S M Sajjadi, Antonio Vergari, Michael Black, and Bernhard Schölkopf. From variational to deterministic autoencoders. arXiv preprint arXiv: 1903.12436, 2019.
[15] Pawe? Gniewek, Bradley Worley, Kate Stafford, Henry van den Bedem, and Brandon Anderson. Learning physics confers pose-sensitivity in structure-based virtual screening. arXiv preprint arXiv: 2110.15459, 2021.
[16] Rafael Gómez-Bombarelli, Jennifer N Wei, David Duvenaud, José Miguel Hernández-Lobato, Benjamin Sánchez-Lengeling, Dennis Sheberla, Jorge Aguilera-Iparraguirre, Timothy D Hirzel, Ryan P Adams, and Alán Aspuru-Guzik. Automatic chemical design using a data-driven continuous representation of molecules. ACS Central Science, 4 (2): 268-276, 2018.
[17] Christoph Gorgulla, Andras Boeszoermenyi, Zi-Fu Wang, Patrick D Fischer, Paul W Coote, Krishna M Padmanabha Das, Yehor S Malets, Dmytro S Radchenko, Yurii S Moroz, David A Scott, et al. An open-source drug discovery platform enables ultra-large virtual screens. Nature, 580 (7805), 2020.
[18] David E Graff, Eugene I Shakhnovich, and Connor W Coley. Accelerating high-throughput virtual screening through molecular pool-based active learning. Chemical Science, 12 (22): 7866-7881, 2021.
[19] Ryan-Rhys Griffiths and José Miguel Hernández-Lobato. Constrained bayesian optimization for automatic chemical design using variational autoencoders. Chemical Science, 11 (2): 577-586, 2020.
[20] Oleksandr O Grygorenko, Dmytro S Radchenko, Igor Dziuba, Alexander Chuprina, Kateryna E Gubina, and Yurii S Moroz. Generating multibillion chemical space of readily accessible screening compounds. Iscience, 23 (11), 2020.
[21] Julien Horwood and Emmanuel Noutahi. Molecular design in synthetically accessible chemical space via deep reinforcement learning. ACS Omega, 5 (51): 32984-32994, 2020.
[22] John J Irwin and Brian K Shoichet. Docking screens for novel ligands conferring new biology: Miniperspective. Journal of Medicinal Chemistry, 59 (9): 4103-4120, 2016.
[23] Wengong Jin, Regina Barzilay, and Tommi Jaakkola. Junction tree variational autoencoder for molecular graph generation. International Conference on Machine Learning, 2018.
[24] Wengong Jin, Regina Barzilay, and Tommi Jaakkola. Hierarchical generation of molecular graphs using structural motifs. International Conference on Machine Learning, pages 4839-4848, 2020.
[25] Wengong Jin, Regina Barzilay, and Tommi Jaakkola. Multi-objective molecule generation using interpretable substructures. International Conference on Machine Learning, 2020.
[26] Nikita Kitaev, ?ukasz Kaiser, and Anselm Levskaya. Reformer: the efficient transformer. arXiv preprint arXiv: 2001.04451, 2020.
[27] Xiangzhe Kong, Zhixing Tan, and Yang Liu. Graphpiece: Efficiently generating high-quality molecular graph with substructures. arXiv preprint arXiv: 2106.15098, 2021.
[28] Mario Krenn, Florian Häse, AkshatKumar Nigam, Pascal Friederich, and Alan Aspuru-Guzik. Self-referencing embedded strings (selfies): A 100% robust molecular string representation. Machine Learning: Science and Technology, 1 (4), 2020.
[29] Matt J Kusner, Brooks Paige, and José Miguel Hernández-Lobato. Grammar variational autoencoder. International Conference on Machine Learning, 2017.
[30] Greg Landrum. RDKit: Open-source cheminformatics, 2006.
[31] Juho Lee, Yoonho Lee, Jungtaek Kim, Adam Kosiorek, Seungjin Choi, and Yee Whye Teh. Set transformer: A framework for attention-based permutation-invariant neural networks. International Conference on Machine Learning, pages 3744-3753, 2019.
[32] Qi Liu, Miltiadis Allamanis, Marc Brockschmidt, and Alexander Gaunt. Constrained graph variational autoencoders for molecule design. Advances in Neural Information Processing Systems, 31, 2018.
[33] Jiankun Lyu, Sheng Wang, Trent E Balius, Isha Singh, Anat Levit, Yurii S Moroz, Matthew J O'Meara, Tao Che, Enkhjargal Algaa, Kateryna Tolmachova, et al. Ultra-large library docking for discovering new chemotypes. Nature, 566 (7743): 224-229, 2019.
[34] Krzysztof Maziarz, Henry Jackson-Flux, Pashmina Cameron, Finton Sirockin, Nadine Schneider, Nikolaus Stiefl, Marwin Segler, and Marc Brockschmidt. Learning to extend molecular scaffolds with structural motifs. arXiv preprint arXiv: 2103.03864, 2021.
[35] Rocio Mercado, Tobias Rastemo, Edvard Lindelöf, Günter Klambauer, Ola Engkvist, Hongming Chen, and Esben Jannik Bjerrum. Graph networks for molecular design. Machine Learning: Science and Technology, 2 (2): 025023, 2021.
[36] Joshua Meyers, Benedek Fabian, and Nathan Brown. De novo molecular design and generative models. Drug Discovery Today, 26 (11): 2707-2715, 2021.
[37] AkshatKumar Nigam, Robert Pollice, Mario Krenn, Gabriel dos Passos Gomes, and Alan Aspuru-Guzik. Beyond generative models: superfast traversal, optimization, novelty, exploration and discovery (stoned) algorithm for molecules using selfies. Chemical Science, 12 (20): 7079-7090, 2021.
[38] Hakime Öztürk, Arzucan Özgür, and Elif Ozkirimli. DeepDTA: deep drug-target binding affinity prediction. Bioinformatics, 34 (17): 1821-1829, 2018.
[39] Joseph M Paggi, Julia A Belk, Scott A Hollingsworth, Nicolas Villanueva, Alexander S Powers, Mary J Clark, Augustine G Chemparathy, Jonathan E Tynan, Thomas K Lau, Roger K Sunahara, et al. Leveraging nonstructural data to predict structures and affinities of protein-ligand complexes. Proceedings of the National Academy of Sciences, 118 (51), 2021.
[40] Pavel Polishchuk. Control of synthetic feasibility of compounds generated with CReM. Journal of Chemical Information and Modeling, 60 (12): 6074-6080, 2020.
[41] Daniil Polykovskiy, Alexander Zhebrak, Benjamin Sanchez-Lengeling, Sergey Golovanov, Oktai Tatanov, Stanislav Belyaev, Rauf Kurbanov, Aleksey Artamonov, Vladimir Aladinskiy, Mark Veselov, et al. Molecular sets (MOSES): a benchmarking platform for molecular generation models. Frontiers in Pharmacology, 11:1931, 2020.
[42] Mariya Popova, Mykhailo Shvets, Junier Oliva, and Olexandr Isayev. Molecularrnn: Generating realistic molecular graphs with optimized properties. arXiv preprint arXiv: 1905.13372, 2019.
[43] Matthew Ragoza, Joshua Hochuli, Elisa Idrobo, Jocelyn Sunseri, and David Ryan Koes. Protein-ligand scoring with convolutional neural networks. Journal of Chemical Information and Modeling, 57 (4): 942-957, 2017.
[44] Arman A Sadybekov, Anastasiia V Sadybekov, Yongfeng Liu, Christos Iliopoulos-Tsoutsouvas, Xi-Ping Huang, Julie Pickett, Blake Houser, Nilkanth Patel, Ngan K Tran, Fei Tong, et al. Synthon-based ligand discovery in virtual libraries of over 11 billion compounds. Nature, 601 (7893): 452-459, 2022.
[45] Robert Schmidt, Raphael Klein, and Matthias Rarey. Maximum common substructure searching in combinatorial make-on-demand compound spaces. Journal of Chemical Information and Modeling, 2021.
[46] Marwin H S Segler, Thierry Kogej, Christian Tyrchan, and Mark P Waller. Generating focused molecule libraries for drug discovery with recurrent neural networks. ACS Central Science, 4 (1): 120-131, 2018.
[47] Brian K Shoichet. Virtual screening of chemical libraries. Nature, 432 (7019): 862-865, 2004.
[48] Martin Simonovsky and Nikos Komodakis. Graphvae: Towards generation of small graphs using variational autoencoders. International Conference on Artificial Neural Networks, 2018.
[49] Casper Kaae Sønderby, Tapani Raiko, Lars Maaløe, Søren Kaae Sønderby, and Ole Winther. Ladder variational autoencoders. Advances in Neural Information Processing Systems, 29, 2016.
[50] Tiago Sousa, João Correia, Vitor Pereira, and Miguel Rocha. Generative deep learning for targeted compound design. Journal of Chemical Information and Modeling, 61 (11): 5343-5361, 2021.
[51] Kate Stafford, Brandon M Anderson, Jon Sorenson, and Henry van den Bedem. AtomNet PoseRanker: Enriching ligand pose quality for dynamic proteins in virtual high-throughput screens. Journal of Chemical Information and Modeling, 62 (5): 1178-1189, 2022.
[52] Hannes Stärk, Octavian-Eugen Ganea, Lagnajit Pattanaik, Regina Barzilay, and Tommi Jaakkola. Equibind: Geometric deep learning for drug binding structure prediction. arXiv preprint arXiv: 2202.05146, 2022.
[53] Kosuke Takeuchi, Ryo Kunimoto, and Jürgen Bajorath. R-group replacement database for medicinal chemistry. Future Science O A, 7 (8): FSO742, 2021.
[54] Xiaochu Tong, Xiaohong Liu, Xiaoqin Tan, Xutong Li, Jiaxin Jiang, Zhaoping Xiong, Tingyang Xu, Hualiang Jiang, Nan Qiao, and Mingyue Zheng. Generative models for de novo drug design. Journal of Medicinal Chemistry, 64 (19): 14011-14027, 2021.
[55] Kenneth E Train. Discrete choice methods with simulation. Cambridge University Press, 2009.
[56] Masashi Tsubaki, Kentaro Tomii, and Jun Sese. Compound-protein interaction prediction with end-to-end learning of neural networks for graphs and sequences. Bioinformatics, 35 (2): 309-318, 2019.
[57] Izhar Wallach, Michael Dzamba, and Abraham Heifets. AtomNet: a deep convolutional neural network for bioactivity prediction in structure-based drug discovery. arXiv preprint arXiv: 1510.02855, 2015.
[58] Wendy A Warr, Marc C Nicklaus, Christos A Nicolaou, and Matthias Rarey. Exploration of ultra-large compound collections for drug discovery. Journal of Chemical Information and Modeling, 62 (9): 2021-2034, 2022.
[59] Robin Winter, Frank Noé, and Djork-Arne Clevert. Permutation-invariant variational autoencoder for graph-level representation learning. Advances in Neural Information Processing Systems, 34, 2021.
[60] Jiaxuan You, Bowen Liu, Zhitao Ying, Vijay Pande, and Jure Leskovec. Graph convolutional policy network for goal-directed molecular graph generation. Advances in Neural Information Processing Systems, 31, 2018.
[61] Manzil Zaheer, Satwik Kottur, Siamak Ravanbakhsh, Barnabas Poczos, Russ R Salakhutdinov, and Alexander J Smola. Deep Sets. Advances in Neural Information Processing Systems, 30, 2017.

### CONCLUSION

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

1. A computer system for querying a combinatorial synthesis library comprising a plurality of compounds, wherein
the combinatorial synthesis library represents a plurality of reaction types,
each respective reaction type in the plurality of reaction types has a corresponding mapping to a corresponding plurality of reactants, and
each respective reactant in each corresponding plurality of reactants has a corresponding mapping to a corresponding plurality of synthons, the computer system comprising:
one or more central processing units;
one or more graphic processing units, wherein each graphic processing unit in the one or more graphic processing units comprises 100 or more cores; and
memory addressable by the one or more central processing units, the memory storing at least one program for execution, at least in part, by the one or more graphic processing units, the at least one program comprising instructions for:
(A) inputting a query, wherein the query is a single graph, into a molecular encoder model, wherein the molecular encoder model comprises a message passing neural network comprising a plurality of message passing layers that collectively comprise a first plurality of parameters, thereby obtaining a query vector by application of the first plurality of parameters to the single graph;
(B) inputting the query vector into a reaction query generator model comprising a second plurality of parameters thereby obtaining, as output from the reaction query generator model, a first reaction type in the plurality of reaction types by application of the second plurality of parameters to the query vector;
(C) determining a corresponding synthon for each respective reactant in a first plurality of reactants corresponding to the first reaction type from among the corresponding plurality of synthons mapped to the respective reactant by inputting the respective reactant into a synthon query generator model comprising a third plurality of parameters thereby obtaining, as output from the synthon query generator model, the corresponding synthon by application of the third plurality of parameters to the respective reactant, thereby determining a set of synthons, each synthon in the set of synthons corresponding to a reactant in the first plurality of reactants; and
(D) identifying a molecular structure in the combinatorial synthesis library that includes the set of synthons arranged in accordance with a synthesis rule associated with the first reaction type.

2. The computer system of claim 1, wherein the reaction query generator model is a two-layer perceptron with intermediate ReLU activation.

3. The computer system of claim 1, wherein the synthon query generator model is a two-layer perceptron with intermediate ReLU activation.

4. The computer system of claim 1, wherein
the first plurality of parameters comprises 100,000 parameters,
the second plurality of parameters comprises 5,000 parameters, and
the third plurality of parameters comprises 5,000 parameters.

5. The computer system of claim 1, wherein
the single graph comprises a plurality of nodes and a plurality of edges, and
each node in the plurality of nodes is connected by at least one edge in the plurality of edges to another node in the plurality of nodes.

6. The computer system of claim 5, wherein each node in the plurality of nodes is associated with:
(i) a corresponding element type in a plurality of element types,
(ii) a node degree in a plurality of node degrees,
(iii) a hybridization in a plurality of hybridizations,
(iv) a number of bonded hydrogens,
(v) a formal charge from among a set of formal charges, and
(vi) a binary indication of aromaticity.

7. The computer system of claim 5, wherein each respective bond in the plurality of bonds is associated with:
(i) a bond type,
(ii) a binary indication of conjugation,
(iii) a binary of indication of whether or not the respective bond is in a ring, and
(iv) an indication of stereochemistry.

8. The computer system of claim 1, wherein the plurality of reaction types comprises 20 or more reaction types and the combinatorial synthesis library comprises 100 or more compounds for each reaction type in the plurality of reaction types.

9. The computer system of claim 1, wherein the first plurality of reactants comprises three or more reactants and the corresponding mapping for the corresponding plurality of synthons for a reactant in the three or more reactants comprises ten or more synthons.

10. The computer system of claim 1, wherein
the output from the reaction query generator model is used to identify a first reaction key in a plurality of reaction keys through a first query key lookup, and
each reaction key in the plurality of reaction keys represents a synthetic reaction that can be used to synthesize one or more compounds in the combinatorial synthesis library.

11. The computer system claim 1, wherein an output from the synthon query generator model is used to identify a synthon key for the corresponding synthon through a second query key lookup.

12. The computer system of claim 1, wherein the single graph represents a single molecular compound present in the combinatorial synthesis library.

13. The computer system of claim 1, wherein the single graph represents a weighted composite of a first graph of a first molecular compound and a second graph of a second molecular compound.

14. The computer system of claim 1, wherein
the single graph represents a weighted composite of a plurality of graphs of a second plurality of compounds, and
the second plurality of compounds have a common property.

15. The computer system of claim 14, wherein the common property is a Tanimoto distance less than a threshold value to each other compound it the second plurality of compounds.

16. The computer system of claim 14, wherein the common property is a binding coefficient to macromolecular target that is less than a threshold value.

17. The computer system of claim 1, wherein the plurality of compounds comprises a billion or more compounds and the molecular structure outputted by the identifying (D) is any one of the billion or more compounds satisfying the query.

18. The computer system of claim 1, wherein the plurality of compounds comprises a trillion or more compounds and the molecular structure outputted by the identifying (D) is any one of the trillion or more compounds satisfying the query.

19. The computer system of claim 1, wherein the single graph represents a query molecular compound as a set of atom features and a set of bond features.

20. The computer system of claim 19, wherein
the set of atom features comprises element type, node degree, hybridization, chirality, bonded hydrogens, formal charge, aromaticity, and
the set of bond features comprises bond time, conjugated, in a ring, and stereochemistry.

21. The computer system of claim 20, wherein each non-hydrogen atom is the query molecular compound is represented by 2000 or more parameters in the set of atom features and each covalent bond in the molecular compound is represented by 500 or more parameters in the set of bond features.

22. A method for querying a combinatorial synthesis library comprising a plurality of compounds, wherein
the combinatorial synthesis library represents a plurality of reaction types,
each respective reaction type in the plurality of reaction types has a corresponding mapping to a corresponding plurality of reactants, and
each respective reactant in each corresponding plurality of reactants has a corresponding mapping to a corresponding plurality of synthons, and
the method performed at a computer system comprising:
one or more central processing units;
one or more graphic processing units, wherein each graphic processing unit in the one or more graphic processing units comprises 100 or more cores; and
memory addressable by the one or more central processing units, the memory storing at least one program for execution, at least in part, by the one or more graphic processing units, the at least one program comprising instructions to perform the method comprising:
(A) inputting a query, wherein the query is a single graph, into a molecular encoder model, wherein the molecular encoder model comprises a message passing neural network comprising a plurality of message passing layers that collectively comprise a first plurality of parameters, thereby obtaining a query vector by application of the first plurality of parameters to the single graph;
(B) inputting the query vector into a reaction query generator model comprising a second plurality of parameters thereby obtaining, as output from the reaction query generator model, a first reaction type in the plurality of reaction types by application of the second plurality of parameters to the query vector;
(C) determining a corresponding synthon for each respective reactant in the first plurality of reactants from among the corresponding plurality of synthons mapped to the respective reactant by inputting the respective reactant into a synthon query generator model comprising a third plurality of parameters thereby obtaining, as output from the synthon query generator model, the corresponding synthon by application of the third plurality of parameters to the respective reactant, thereby determining a set of synthons, each synthon in the set of synthons corresponding to a reactant in the first plurality of reactants; and
(D) identifying a molecular structure in the combinatorial synthesis library that includes the set of synthons arranged in accordance with a synthesis rule associated with the first reaction type.

23. A computer readable storage medium storing one or more programs, the one or more programs comprising instructions, which when executed by a computer system with one or more central processing units and one or more graphic processing units, wherein each graphic processing unit in the one or more graphic processing units comprises 100 or more cores, and a memory that causes the computer system to query a combinatorial synthesis library, wherein
the combinatorial synthesis library represents a plurality of reaction types,
each respective reaction type in the plurality of reaction types has a corresponding mapping to a corresponding plurality of reactants, and
each respective reactant in each corresponding plurality of reactants has a corresponding mapping to a corresponding plurality of synthons, the
the query of the combinatorial synthesis library performed at least in part by the one or more graphic processing units, by a method comprising:
(A) inputting a query, wherein the query is a single graph, into a molecular encoder model, wherein the molecular encoder model comprises a message passing neural network comprising a plurality of message passing layers that collectively comprise a first plurality of parameters, thereby obtaining a query vector by application of the first plurality of parameters to the single graph;
(B) inputting the query vector into a reaction query generator model comprising a second plurality of parameters thereby obtaining, as output from the reaction query generator model, a first reaction type in the plurality of reaction types by application of the second plurality of parameters to the query vector;
(C) determining a corresponding synthon for each respective reactant in the first plurality of reactants from among the corresponding plurality of synthons mapped to the respective reactant by inputting the respective reactant into a synthon query generator model comprising a third plurality of parameters thereby obtaining, as output from the synthon query generator model, the corresponding synthon by application of the third plurality of parameters to the respective reactant, thereby determining a set of synthons, each synthon in the set of synthons corresponding to a reactant in the first plurality of reactants; and
(D) identifying a molecular structure in the combinatorial synthesis library that includes the set of synthons arranged in accordance with a synthesis rule associated with the first reaction type.

24-44. (canceled)

45. A method for querying a combinatorial synthesis library comprising a plurality of compounds, wherein
the combinatorial synthesis library represents a plurality of reaction types,
each respective reaction type in the plurality of reaction types has a corresponding mapping to a corresponding plurality of reactants, and
each respective reactant in each corresponding plurality of reactants has a corresponding mapping to a corresponding plurality of synthons, and
the method performed at a computer system comprising:
one or more processing units;
memory addressable by the one or more processing units, the memory storing at least one program for execution by the one or more processing units, the at least one program comprising instructions to perform a method comprising:
(A) inputting a query, wherein the query is an arbitrary graph, into a molecular encoder model comprising a first plurality of parameters, thereby obtaining a query vector by application of the first plurality of parameters to the arbitrary graph;
(B) inputting the query vector into a reaction query generator model comprising a second plurality of parameters thereby obtaining, as output from the reaction query generator model, a first reaction type in the plurality of reaction types by application of the second plurality of parameters to the query vector;
(C) determining a corresponding synthon for each respective reactant in the first plurality of reactants from among the corresponding plurality of synthons mapped to the respective reactant by inputting the respective reactant into a synthon query generator model comprising a third plurality of parameters thereby obtaining, as output from the synthon query generator model, the corresponding synthon by application of the third plurality of parameters to the respective reactant, thereby determining a set of synthons, each synthon in the set of synthons corresponding to a reactant in the first plurality of reactants; and
(D) identifying a molecular structure in the combinatorial synthesis library that includes the set of synthons arranged in accordance with a synthesis rule associated with the first reaction type.

45B. (canceled)
